SEQUENCE LISTING (93 sequences)

SEQ ID NO: 1 - protein, 759 aa, Homo sapiens
GPRAPCAAACTCAGDSLDCGGRGLAALPGDLPSWTRSLNLSYNKLSEIDPAGFEDLPNLQEVYLNNNELTAVPSLGAASSHVVSLFLQHNKIRSVEGSQLKAYLSLEVLDLSLNNITEVRNTCFPHGPPIKELNLAGNRIGTLELGAFDGLSRSLLTLRLSKNRITQLPVRAFKLPRLTQLDLNRNRIRLIEGLTFQGLNSLEVLKLQRNNISKLTDGAFWGLSKMHVLHLEYNSLVEVNSGSLYGLTALHQLHLSNNSIARIHRKGWSFCQKLHELVLSFNNLTRLDEESLAELSSLSVLRLSHNSISHIAEGAFKGLRSLRVLDLDHNEISGTIEDTSGAFSGLDSLSKLTLFGNKIKSVAKRAFSGLEGLEHLNLGGNAIRSVQFDAFVKMKNLKELHISSDSFLCDCQLKWLPPWLIGRMLQAFVTATCAHPESLKGQSIFSVPPESFVCDDFLKPQIITQPETTMAMVGKDIRFTCSAASSSSSPMTFAWKKDNEVLTNADMENFVHVHAQDGEVMEYTTILHLRQVTFGHEGRYQCVITNHFGSTYSHKARLTVNVLPSFTKTPHDITIRTTTVARLECAATGHPNPQIAWQKDGGTDFPAARERRMHVMPDDDVFFITDVKIDDAGVYSCTAQNSAGSISANATLTVLETPSLVVPLEDRVVSVGETVALQCKATGNPPPRITWFKGDRPLSLTERHHLTPDNQLLVVQNVVAEDAGRYTCEMSNTLGTERAHSQLSVLPAAGCRKDGTTVG

SEQ ID NO: 2 - DNA, 2397 bp, Homo sapiens
ggcccgcgggcgccctgcgcggccgcctgcacttgcgctggggactcgctggactgcggtgggcgcgggctggctgcgttgcccggggacctgccctcctggacgcggagcctaaacctgagttacaacaaactctctgagattgaccctgctggttttgaggacttgccgaacctacaggaagtgtacctcaataataatgagttgacagcggtaccatccctgggcgctgcttcatcacatgtcgtctctctctttctgcagcacaacaagattcgcagcgtggaggggagccagctgaaggcctacctttccttagaagtgttagatctgagtttgaacaacatcacggaagtgcggaacacctgctttccacacggaccgcctataaaggagctcaacctggcaggcaatcggattggcaccctggagttgggagcatttgatggtctgtcacggtcgctgctaactcttcgcctgagcaaaaacaggatcacccagcttcctgtaagagcattcaagctacccaggctgacacaactggacctcaatcggaacaggattcggctgatagagggcctcaccttccaggggctcaacagcttggaggtgctgaagcttcagcgaaacaacatcagcaaactgacagatggggccttctggggactgtccaagatgcatgtgctgcacctggagtacaacagcctggtagaagtgaacagcggctcgctctacggcctcacggccctgcatcagctccacctcagcaacaattccatcgctcgcattcaccgcaagggctggagcttctgccagaagctgcatgagttggtcctgtccttcaacaacctgacacggctggacgaggagagcctggccgagctgagcagcctgagtgtcctgcgtctcagccacaattccatcagccacattgcggagggtgccttcaagggactcaggagcctgcgagtcttggatctggaccataacgagatttcgggcacaatagaggacacgagcggcgccttctcagggctcgacagcctcagcaagctgactctgtttggaaacaagatcaagtctgtggctaagagagcattctcggggctggaaggcctggagcacctgaaccttggagggaatgcgatcagatctgtccagtttgatgcctttgtgaagatgaagaatcttaaagagctccatatcagcagcgacagcttcctgtgtgactgccagctgaagtggctgcccccgtggctaattggcaggatgctgcaggcctttgtgacagccacctgtgcccacccagaatcactgaagggtcagagcattttctctgtgccaccagagagtttcgtgtgcgatgacttcctgaagccacagatcatcacccagccagaaaccaccatggctatggtgggcaaggacatccggtttacatgctcagcagccagcagcagcagctcccccatgacctttgcctggaagaaagacaatgaagtcctgaccaatgcagacatggagaactttgtccacgtccacgcgcaggacggggaagtgatggagtacaccaccatcctgcacctccgtcaggtcactttcgggcacgagggccgctaccaatgtgtcatcaccaaccactttggctccacctattcacataaggccaggctcaccgtgaatgtgttgccatcattcaccaaaacgccccacgacataaccatccggaccaccaccgtggcccgcctcgaatgtgctgccacaggtcacccaaaccctcagattgcctggcagaaggatggaggcacggatttccccgctgcccgtgagcgacgcatgcatgtcatgccggatgacgacgtgtttttcatcactgatgtgaaaatagatgacgcaggggtttacagctgtactgctcagaactcagccggttctatttcagctaatgccaccctgactgtcctagagaccccatccttggtggtccccttggaagaccgtgtggtatctgtgggagaaacagtggccctccaatgcaaagccacggggaaccctccgccccgcatcacctggttcaagggggaccgcccgctgagcctcactgagcggcaccacctgacccctgacaaccagctcctggtggttcagaacgtggtggcagaggatgcgggccgatatacctgtgagatgtccaacaccctgggcacggagcgagctcacagccagctgagcgtcctgcccgcagcaggctgcaggaaggatgggaccacggtaggcatcttcaccattgctgtcgtgagcagcatcgtcctgacgtcactggtctgggtgtgcatcatctaccagaccaggaagaagagtgaagagtacagtgtcaccaacacagatgaaaccgtc

SEQ ID NO: 3 - protein, 761 aa, Mus musculus
QAGPRAPCAAACTCAGDSLDCSGRGLATLPRDLPSWTRSLNLSYNRLSEIDSAAFEDLTNLQEVYLNSNELTAIPSLGAASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTTVG

SEQ ID NO: 4 - DNA, 2283 bp, Mus musculus
caggctggcccgcgggccccctgcgcggccgcctgcacttgcgccggggactcgctggactgcagtgggcgcgggctggcgacgctgccccgggacctgccctcctggacgcgcagcctaaacctgagttataacagactctccgagatcgactctgctgcttttgaggacttgacgaatctgcaggaagtgtacctcaacagcaatgagctgacagccataccatcactgggcgctgcttccataggagttgtctctctctttttgcagcacaacaagatccttagtgtggatgggagccagctgaagtcgtacctgtccttggaagtgctggatctgagttccaacaacatcacggaaattcggagctcctgtttcccgaacggcctgcgtataagggaactcaacttggcgagcaaccgcatcagcatcctggagtctggagcatttgatggtctgtcgcggtcactgctgactctccgtctgagcaaaaacaggatcacccagcttcctgtgaaagcgttcaagctacccaggctgacacaactagacctgaatcggaatcggattcggctgattgaaggcctcacgttccaggggctcgacagcttagaggtgctgaggcttcagaggaacaacatcagcaggctgacggacggggccttctgggggctgtctaagatgcacgtgctgcacctggagtacaacagtctggtggaagtgaacagtggctccctctatggcctcacagccctgcaccagctgcacctcagcaacaactccatctctcgaattcagcgtgatggctggagcttctgccaaaagctgcatgagttgattctgtccttcaacaacctcacgcggctggatgaggagagtctagcggagttgagcagcctcagtatcctgcgcctcagtcacaacgccatcagtcacattgctgaaggcgccttcaagggactcaagagtctgcgggtcttggacctggaccataacgagatctcgggtacaatcgaggataccagtggtgcctttacggggcttgacaacctcagcaagctgactctgtttggaaacaagatcaaatctgtggctaagagagccttctcgggcctggaaagcctggaacacctgaaccttggagagaatgcaatcaggtctgtccagtttgatgcctttgcaaagatgaagaaccttaaagagctctacatcagcagtgagagcttcctgtgtgactgccagctcaagtggctgcccccatggctaatgggtaggatgctgcaggcctttgtgacagccacctgtgcccatccagagtcgctgaagggccagagcattttctcagtgctgccagacagctttgtgtgtgatgactttccaaagccacagatcatcacccagcctgagacgaccatggctgtggtgggcaaggacatccgtttcacatgctccgcagccagcagcagcagctcaccaatgaccttcgcctggaagaaggacaatgaggtcctggccaatgcagacatggagaactttgcccacgtccgtgcacaggacggcgaagtgatggagtataccactatcctgcacctccgtcacgtcacctttgggcacgagggccgctaccagtgtatcatcacaaaccactttggctccacatactcccacaaagccaggctcactgtgaatgtgttgccatcattcactaaaataccccatgacattgccatccggactggcaccacagcccgcctcgagtgtgctgccacgggccaccctaaccctcagattgcctggcagaaggatggaggcaccgatttcccggcagctcgtgagcgacgcatgcatgttatgccagacgatgatgtgttcttcatcactgatgtgaaaatagacgacatgggggtctacagctgcactgcccagaactcggcaggctcggtttcagccaacgctaccctcacagtcttagaaactccatccttggcagtgcctctggaagaccgtgtggtaactgtgggagaaacagtggccttccagtgcaaagcaaccgggagccccacaccacgcatcacctggcttaagggaggtcgcccattgagcctcacagagcgccaccatttcactccaggcaaccagctgctggttgttcagaatgtgatgatagacgatgcagggcggtatacctgtgagatgtctaatcccctgggcactgagcgagcacatagccagctgagcattttacctacccctggctgccggaaggatgggaccaccgtaggc

SEQ ID NO: 5 - protein, 5 aa, Artificial Sequence
  A7 heavy chain CDR 1
GYDMS

SEQ ID NO: 6 - protein, 9 aa, Artificial Sequence
  A7 heavy chain CDR 2
LIYPDSGNK

SEQ ID NO: 7 - protein, 13 aa, Artificial Sequence
  A7 heavy chain CDR 3
RDAGLSWAGAFDY

SEQ ID NO: 8 - protein, 12 aa, Artificial Sequence
  A7 light chain CDR 1
SGSSSNIGSNYV

SEQ ID NO: 9 - protein, 4 aa, Artificial Sequence
  A7 light chain CDR 2
SDSH

SEQ ID NO: 10 - protein, 9 aa, Artificial Sequence
  A7 light chain CDR 3
GSWDYSLSA

SEQ ID NO: 11 - protein, 121 aa, Artificial Sequence
  A7 heavy chain_variable region
EVQLLESGGGLVQPGGSLRLSCAASGFTFSGYDMSWVRQAPGKGLEWVSLIYPDSGNKYYADSVKGRFTISRDNSKNTLYLQMNSLRAEDTAVYYCARDAGLSWAGAFDYWGQGTLVTVSS

SEQ ID NO: 12 - protein, 110 aa, Artificial Sequence
  A7 light chain_variable region
QSVLTQPPSASGTPGQRVTISCSGSSSNIGSNYVTWYQQLPGTAPKLLIYSDSHRPSGVPDRFSGSKSGTSASLAISGLQSEDEADYYCGSWDYSLSAYVFGGGTKLTVL

SEQ ID NO: 13 - protein, 5 aa, Artificial Sequence
  C8 heavy chain CDR 1
NYYMS

SEQ ID NO: 14 - protein, 8 aa, Artificial Sequence
  C8 heavy chain CDR 2
GISPGDSS

SEQ ID NO: 15 - protein, 13 aa, Artificial Sequence
  C8 heavy chain CDR 3
KGLYSNPNEPFDY

SEQ ID NO: 16 - protein, 13 aa, Artificial Sequence
  C8 light chain CDR 1
TGSSSNIGSNYVS

SEQ ID NO: 17 - protein, 4 aa, Artificial Sequence
  C8 light chain CDR 2
DDSQ

SEQ ID NO: 18 - protein, 9 aa, Artificial Sequence
  C8 light chain CDR 3
GTWDYSLNG

SEQ ID NO: 19 - protein, 121 aa, Artificial Sequence
  C8 heavy chain_variable region
EVQLLESGGGLVQPGGSLRLSCAASGFTFSNYYMSWVRQAPGKGLEWVSGISPGDSSTYYADSVKGRFTISRDNSKNTLYLQMNSLRAEDTAVYYCAKGLYSNPNEPFDYWGQGTLVTVSS

SEQ ID NO: 20 - protein, 110 aa, Artificial Sequence
  C8 light chain_variable region
QSVLTQPPSASGTPGQRVTISCTGSSSNIGSNYVSWYQQLPGTAPKLLIYDDSQRPSGVPDRFSGSKSGTSASLAISGLRSEDEADYYCGTWDYSLNGYVFGGGTKLTVL

SEQ ID NO: 21 - protein, 5 aa, Artificial Sequence
  E7 heavy chain CDR 1
SYDMS

SEQ ID NO: 22 - protein, 9 aa, Artificial Sequence
  E7 heavy chain CDR 2
GISPDGSNI

SEQ ID NO: 23 - protein, 19 aa, Artificial Sequence
  E7 heavy chain CDR 3
KVGLRCRYEACSYAYGMDV

SEQ ID NO: 24 - protein, 13 aa, Artificial Sequence
  E7 light chain CDR 1
SGSSSNIGSNYVS

SEQ ID NO: 25 - protein, 4 aa, Artificial Sequence
  E7 light chain CDR 2
SDSH

SEQ ID NO: 26 - protein, 9 aa, Artificial Sequence
  E7 light chain CDR 3
ATWDSSLNG

SEQ ID NO: 27 - protein, 127 aa, Artificial Sequence
  E7 heavy chain_variable region
EVQLLESGGGLVQPGGSLRLSCAASGFTFSSYDMSWVRQAPGKGLEWVSGISPDGSNIYYADSVKGRFTISRDNSKNTLYLQMNSLRAEDTAVYYCAKVGLRCRYEACSYAYGMDVWGQGTLVTVSS

SEQ ID NO: 28 - protein, 110 aa, Artificial Sequence
  E7 light chain_variable region
QSVLTQPPSASGTPGQRVTISCSGSSSNIGSNYVSWYQQLPGTAPKLLIYSDSHRPSGVPDRFSGSKSGTSASLAISGLRSEDEADYYCATWDSSLNGYVFGGGTKLTVL

SEQ ID NO: 29 - protein, 5 aa, Artificial Sequence
  G3 heavy chain CDR 1
NYDMS

SEQ ID NO: 30 - protein, 9 aa, Artificial Sequence
  G3 heavy chain CDR 2
SISPSSGSI

SEQ ID NO: 31 - protein, 13 aa, Artificial Sequence
  G3 heavy chain CDR 3
KDLDAFWRPSFDY

SEQ ID NO: 32 - protein, 13 aa, Artificial Sequence
  G3 light chain CDR 1
TGSSSNIGNNNVN

SEQ ID NO: 33 - protein, 4 aa, Artificial Sequence
  G3 light chain CDR 2
SDSH

SEQ ID NO: 34 - protein, 9 aa, Artificial Sequence
  G3 light chain CDR 3
GSWDDSLSA

SEQ ID NO: 35 - protein, 121 aa, Artificial Sequence
  G3 heavy chain_variable region
EVQLLESGGGLVQPGGSLRLSCAASGFTFSNYDMSWVRQAPGKGLEWVSSISPSSGSIYYADSVKGRFTISRDNSKNTLYLQMNSLRAEDTAVYYCAKDLDAFWRPSFDYWGQGTLVTVSS

SEQ ID NO: 36 - protein, 110 aa, Artificial Sequence
  G3 light chain_variable region
QSVLTQPPSASGTPGQRVTISCTGSSSNIGNNNVNWYQQLPGTAPKLLIYSDSHRPSGVPDRFSGSKSGTSASLAISGLRSEDEADYYCGSWDDSLSAYVFGGGTKLTVL

SEQ ID NO: 37 - protein, 328 aa, Mus musculus
TTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 38 - protein, 107 aa, Mus musculus
RTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 39 - protein, 330 aa, homo sapiens [sequence omitted]

SEQ ID NO: 40 - protein, 107 aa, Homo sapiens [sequence omitted]

SEQ ID NO: 41 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 42 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 43 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 44 - protein, 245 aa, Artificial Sequence
  Hybrid Fc_Heavy region
RNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 45 - protein, 449 aa, Artificial Sequence
  A7 heavy chain_mouse IgG2 Fc_full sequence
EVQLLESGGGLVQPGGSLRLSCAASGFTFSGYDMSWVRQAPGKGLEWVSLIYPDSGNKYYADSVKGRFTISRDNSKNTLYLQMNSLRAEDTAVYYCARDAGLSWAGAFDYWGQGTLVTVSSTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 46 - protein, 217 aa, Artificial Sequence
  A7 light chain_mouse IgG2 Fc_full sequence
QSVLTQPPSASGTPGQRVTISCSGSSSNIGSNYVTWYQQLPGTAPKLLIYSDSHRPSGVPDRFSGSKSGTSASLAISGLQSEDEADYYCGSWDYSLSAYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 47 - protein, 449 aa, Artificial Sequence
  C8 heavy chain_mouse IgG2 Fc_full sequence
EVQLLESGGGLVQPGGSLRLSCAASGFTFSNYYMSWVRQAPGKGLEWVSGISPGDSSTYYADSVKGRFTISRDNSKNTLYLQMNSLRAEDTAVYYCAKGLYSNPNEPFDYWGQGTLVTVSSTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 48 - protein, 217 aa, Artificial Sequence
  C8 light chain_mouse IgG2 Fc_full sequence
QSVLTQPPSASGTPGQRVTISCTGSSSNIGSNYVSWYQQLPGTAPKLLIYDDSQRPSGVPDRFSGSKSGTSASLAISGLRSEDEADYYCGTWDYSLNGYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 49 - protein, 455 aa, Artificial Sequence
  E7 heavy chain_mouse IgG2 Fc_full sequence
EVQLLESGGGLVQPGGSLRLSCAASGFTFSSYDMSWVRQAPGKGLEWVSGISPDGSNIYYADSVKGRFTISRDNSKNTLYLQMNSLRAEDTAVYYCAKVGLRCRYEACSYAYGMDVWGQGTLVTVSSTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 50 - protein, 217 aa, Artificial Sequence
  E7 light chain_mouse IgG2 Fc_full sequence
QSVLTQPPSASGTPGQRVTISCSGSSSNIGSNYVSWYQQLPGTAPKLLIYSDSHRPSGVPDRFSGSKSGTSASLAISGLRSEDEADYYCATWDSSLNGYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 51 - protein, 449 aa, Artificial Sequence
  G3 heavy chain_mouse IgG2 Fc_full sequence
EVQLLESGGGLVQPGGSLRLSCAASGFTFSNYDMSWVRQAPGKGLEWVSSISPSSGSIYYADSVKGRFTISRDNSKNTLYLQMNSLRAEDTAVYYCAKDLDAFWRPSFDYWGQGTLVTVSSTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 52 - protein, 217 aa, Artificial Sequence
  G3 light chain_mouse IgG2 Fc_full sequence
QSVLTQPPSASGTPGQRVTISCTGSSSNIGNNNVNWYQQLPGTAPKLLIYSDSHRPSGVPDRFSGSKSGTSASLAISGLRSEDEADYYCGSWDDSLSAYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 53 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - DNA, 24 bp, Artificial Sequence
  Mouse Lrig-1 forward primer
gacggaattcagtgaggagaacct

SEQ ID NO: 55 - DNA, 24 bp, Artificial Sequence
  Mouse Lrig-1 reverse primer
caactggtagtggcagcttgtagg

SEQ ID NO: 56 - DNA, 24 bp, Artificial Sequence
  Mouse Lrig-2 forward primer
tcacaaggaacattgtctgaacca

SEQ ID NO: 57 - DNA, 24 bp, Artificial Sequence
  Mouse Lrig-2 reverse primer
gcctgatctaacacatcctcctca

SEQ ID NO: 58 - DNA, 24 bp, Artificial Sequence
  Mouse Lrig-3 forward primer
cagcaccttgagctgaacagaaac

SEQ ID NO: 59 - DNA, 24 bp, Artificial Sequence
  Mouse Lrig-3 reverse primer
ccagcctttggtaatctcggttag

SEQ ID NO: 60 - DNA, 24 bp, Artificial Sequence
  Mouse FOXP3 forward primer
ctttcacctatcccacccttatcc

SEQ ID NO: 61 - DNA, 24 bp, Artificial Sequence
  Mouse FOXP3 reverse primer
attcatctacggtccacactgctc

SEQ ID NO: 62 - DNA, 20 bp, Artificial Sequence
  ACTG1 forward primer
ggcgtcatggtgggcatggg

SEQ ID NO: 63 - DNA, 20 bp, Artificial Sequence
  ACTG1 reverse primer
atggcgtggggaagggcgta

SEQ ID NO: 64 - protein, 48 aa, Artificial Sequence
  Lrig-1 epitope
GDSLDCGGRGLAALPGDLPSSTRSLNLSYNKLSEIDPAGFEDLPNLQE

SEQ ID NO: 65 - protein, 48 aa, Artificial Sequence; identical to SEQ ID NO: 64
  Lrig-1 epitope

SEQ ID NO: 66 - protein, 7 aa, Artificial Sequence
  Lrig-1 epitope
NNNELTA

SEQ ID NO: 67 - protein, 18 aa, Artificial Sequence
  Lrig-1 epitope
NAIRSVQFDAFVKMKNLK

SEQ ID NO: 68 - protein, 5 aa, Artificial Sequence
  Lrig-1 epitope
NKIRS

SEQ ID NO: 69 - protein, 6 aa, Artificial Sequence
  Lrig-1 epitope
VEVNSG

SEQ ID NO: 70 - protein, 6 aa, Artificial Sequence
  Lrig-1 epitope
ARIHRK

SEQ ID NO: 71 - protein, 9 aa, Artificial Sequence
  Lrig-1 epitope
NNITEVRNT

SEQ ID NO: 72 - protein, 14 aa, Artificial Sequence
  Lrig-1 epitope
IRLIEGLTFQGLNS

SEQ ID NO: 73 - protein, 14 aa, Artificial Sequence
  Lrig-1 epitope
ISKLTDGAFWGLSK

SEQ ID NO: 74 - protein, 13 aa, Artificial Sequence
  Lrig-1 epitope
NRITQLPVRAFKL

SEQ ID NO: 75 - protein, 6 aa, Artificial Sequence
  Lrig-1 epitope
KAYLSL

SEQ ID NO: 76 - protein, 25 aa, Artificial Sequence
  Lrig-1 epitope
KKDNEVLTNADMENFVHVHAVMEYT

SEQ ID NO: 77 - protein, 11 aa, Artificial Sequence
  Lrig-1 epitope
SAASSSSSPMT

SEQ ID NO: 78 - protein, 18 aa, Artificial Sequence
  Lrig-1 epitope
FKGDRPLSLTERHHLTPD

SEQ ID NO: 79 - protein, 10 aa, Artificial Sequence
  Lrig-1 epitope
QLSVLLENLY

SEQ ID NO: 80 - protein, 8 aa, Artificial Sequence
  Lrig-1 epitope
KPQIITQP

SEQ ID NO: 81 - protein, 15 aa, Artificial Sequence
  Lrig-1 epitope
VPLEDRVVSVGETVA

SEQ ID NO: 82 - protein, 10 aa, Artificial Sequence
  Lrig-1 epitope
HVMPDDDVFF

SEQ ID NO: 83 - protein, 12 aa, Artificial Sequence
  Lrig-1 epitope
ITNHFGSTYSHK

SEQ ID NO: 84 - protein, 15 aa, Artificial Sequence
  Lrig-1 epitope
QLLVVQNVVAEDAGR

SEQ ID NO: 85 - protein, 8 aa, Artificial Sequence
  Lrig-1 epitope
HLRQVTFG

SEQ ID NO: 86 - protein, 455 aa, Artificial Sequence
  A8 heavy chain_mouse IgG2 Fc_full sequence
EVQLLESGGGLVQPGGSLRLSCAASGFTFSDYDMSWVRQVPGKGLEWVSWISHGGGSIYYADSVKGRFTISRDNSKNTLYLQMNSLRAEDTAVYYCARGLGLCKTGLCYYYDAMDVWGQGTLVTVSSTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 87 - protein, 217 aa, Artificial Sequence
  A8 light chain_mouse IgG2 Fc_full sequence
QSVLTQPPSASGTPGQRVTISCTGSSSNIGNNSVTWYQQLPGTAPKLLIYADNNRPSGVPDRFSGSKSGTSASLAISGLRSEDEADYYCAAWDSSLSAYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 88 - protein, 444 aa, Artificial Sequence
  B8 heavy chain_mouse IgG2 Fc_full sequence
EVQLLESGGGLVQPGGSLRLSCAASGFTFSDYYMSWVRQAPGKGLEWVSGISHDSGSKYYADSVKGRFTISRDNSKNTLYLQMNSLRAEDTAVYYCARHWTTFDYWGQGTLVTVSSTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 89 - protein, 217 aa, Artificial Sequence
  B8 light chain_mouse IgG2 Fc_full sequence
QSVLTQPPSASGTPGQRVTISCSGSSSNIGSNNVTWYQQLPGTAPKLLIYANSNRPSGVPDRFSGSKSGTSASLAISGLRSEDEADYYCGAWDYSLSAYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 90 - protein, 455 aa, Artificial Sequence
  D9 heavy chain_mouse IgG2 Fc_full sequence
EVQLLESGGGLVQPGGSLRLSCAASGFTFSNYAMSWVRQAPGKGLEWVSAIYPGGGSIYYADSVKGRFTISRDNSKNTLYLQMNSLRAEDTAVYYCARDILPCPWGRCYYDYAMDVWGQGTLVTVSSTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 91 - protein, 217 aa, Artificial Sequence
  D9 light chain_mouse IgG2 Fc_full sequence
QSVLTQPPSASGTPGQRVTISCSDSSSNIGSNTVSWYQQLPGTAPKLLIYADNNRPSGVPDRFSGSKSGTSASLAISGLRSEDEADYYCGTWDYSLSGYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 92 - protein, 455 aa, Artificial Sequence
  H6 heavy chain_mouse IgG2 Fc_full sequence
EVQLLESGGGLVQPGGSLRLSCAASGFTFSNYAMSWVRQAPGKGLEWVSVISHGGGSTYYADSVKGRFTISRDNSKNTLYLQMNSLRAEDTAVYYCARVISNCHLGVCYYSNGMDVWGQGTLVTVSSTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 93 - protein, 217 aa, Artificial Sequence
  H6 light chain_mouse IgG2 Fc_full sequence
QSVLTQPPSASGTPGQRVTISCSGSSSNIGNNDVYWYQQLPGTAPKLLIYSDSQRPSGVPDRFSGSKSGTSASLAISGLRSEDEADYYCGTWDYSLSGYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

(19) United States
(12) Patent Application Publication
(10) Pub. No.: US 2022/0275080 A1
(43) Pub. Date: Sep. 1, 2022

(54) BINDING MOLECULE SPECIFIC TO LRIG-1 PROTEIN, AND USE THEREOF

(71) Applicant: GOOD T CELLS, INC., Seoul (KR)

(72) Inventors: Jung Ho Kim, Seoul (KR); Beom Seok Kim, Seoul (KR)

(73) Assignee: GOOD T CELLS, INC., Seoul (KR)

(21) Appl. No.: 17/286,424

(22) PCT Filed: Oct. 17, 2019
(86) PCT No.: PCT/KR2019/013666

§ 371 Date: Apr. 16, 2021

(30) Foreign Application Priority Data
Oct. 17, 2018 (KR) 10-2018-0123858

(51) Int. Cl. C07K 16/28; A61K 47/68; A61P 35/00

(52) U.S. Cl. CPC C07K 16/2803; A61K 47/6801; A61P 35/00; A61K 2039/505

(57) ABSTRACT

Provided herein is a binding molecule capable of specifically binding to LRIG-1 protein, which is located on the surface of a regulatory T cell. Also provided is pharmaceutical composition comprising the binding molecule. Further provided are methods of treating an immune-related disease using the binding molecule or pharmaceutical composition disclosed herein.

### CROSS-REFERENCE TO RELATED APPLICATIONS

This application is U.S. national phase application under 35 U.S.C. § 371 of International Application No. PCT/KR2019/013666, filed on Oct. 17, 2019, which claims the benefit of priority to Korean Patent Application No. KR10-2018-0123858, filed on Oct. 17, 2018, the contents of each of which are incorporated herein by reference in their entirety.

### SEQUENCE LISTING

The ASCII file, entitled corrected SL.txt, created on Oct. 31, 2021, comprising 102,000 bytes, submitted herewith is incorporated herein by reference. The corrected Sequence Listing contains the same sequence information forming part of the original application.

### TECHNICAL FIELD

The present invention relates to a binding molecule capable of specifically binding to leucine-rich and immunoglobulin-like domains 1 (Lrig-1) protein, which is a protein present on the surface of regulatory T cells (Treg cells), and a use thereof.

### BACKGROUND ART

One of the most important traits in all normal individuals is to have the ability to recognize and eliminate non-self antigens, while not detrimentally responding to antigenic substances that make up the self. As such, non-response of the living body to self antigens is called immunologic unresponsiveness or tolerance. Self-tolerance occurs by eliminating lymphocytes that may have specific receptors for self antigens, or by self-inactivation of the ability to respond after contacting self antigens. In a case where a problem arises in inducing or maintaining self-tolerance, an immune response to self antigens occurs, and the disease resulting therefrom is called an autoimmune disease.

For the treatment of the autoimmune disease, a concept of suppressor T cells suggesting the possibility of presence of T cells capable of controlling and suppressing the effector function of conventional T cells was introduced and presented for the first time by Gershon in the early 1970s (R. K. Gershon and K. Kondo, Immunology, 1970, 18: 723-37). Since then, studies have been conducted to elucidate biological properties and functions of regulatory T cells in many areas of immunology.

In this connection, it has been reported that the regulatory T cells (Treg cells) play an important role in naturally preventing occurrence of excessive inflammation and immune responses; however, in a case where an autoimmune disease and a chronic inflammatory disease occur, the function and the number of the regulatory T cells are remarkably decreased. Therefore, in a case of patients with immune and inflammatory diseases, it is important that the regulatory T cells are produced at a normal level, which can be one of the treatments for these diseases.

Until now, studies on genes and proteins which are present specifically in regulatory T cells have been conducted, and it has been presented that substances such as CD25, CTLA4, CD62L, CD38, CD103, GITR, and CD45RB may correspond to marker substances. However, there are no genes and proteins that can target only the regulatory T cells alone.

On the other hand, there are three hypervariable regions called complementarity determining regions (hereinafter referred to as “CDRs”) and four framework regions. The CDRs primarily serve to bind to an epitope on an antigen. The CDRs of each chain are typically referred to as CDR1, CDR2, and CDR3 sequentially starting from the N-terminus, and are also distinguished by the chain where particular CDRs are located.

### Technical Problem

An object of the present invention is to provide a binding molecule specific for Lrig-1 protein present on the surface of regulatory T cells (Treg cells).

Another object of the present invention is to provide a nucleic acid molecule which encodes the binding molecule according to the present invention.

Yet another object of the present invention is to provide an expression vector into which the nucleic acid molecule according to the present invention is inserted.

Still yet another object of the present invention is to provide a host cell line transfected with the expression vector according to the present invention.

Still yet another object of the present invention is to provide an antibody-drug conjugate according to the present invention.

Still yet another object of the present invention is to provide a pharmaceutical composition for preventing or treating an immune-related disease, comprising the binding molecule according to the present invention.

Still yet another object of the present invention is to provide a method for preventing or treating an immune-related disease, comprising a step of administering, to an individual, a pharmaceutically effective amount of the binding molecule or the antibody-drug conjugate (ADC) provided in the present invention.

However, the technical problem to be solved by the present invention is not limited to the above-mentioned problems, and other problems which are not mentioned will be clearly understood by those skilled in the art from the following description.

### Solution to Problem

The present inventors have discovered Lrig-1 protein that is present specifically on the surface of regulatory T cells, have selected an epitope on the protein, and have produced a monoclonal antibody capable of specifically binding to the Lrig-1 protein, thereby completing the present invention.

According to an embodiment of the present invention, there is provided a binding molecule which specifically binds to leucine-rich and immunoglobulin-like domains 1 (Lrig-1) protein.

As used herein, the term “binding molecule” refers to a variable domain comprising an intact immunoglobulin that includes a monoclonal antibody, such as a chimeric, humanized, or human monoclonal antibody, or an immunoglobulin that binds to an antigen, for example, an immunoglobulin fragment that competes with intact immunoglobulins for binding to monomeric HA or trimeric HA of influenza A virus. Regardless of the structure, an antigen-binding fragment binds to the same antigen recognized by intact immunoglobulins. The antigen-binding fragment may include a peptide or polypeptide which contains, out of the amino acid sequence of the binding molecule, an amino acid sequence of two or more contiguous residues, 20 or more contiguous amino acid residues, 25 or more contiguous amino acid residues, 30 or more contiguous amino acid residues, 35 or more contiguous amino acid residues, 40 or more contiguous amino acid residues, 50 or more contiguous amino acid residues, 60 or more contiguous amino acid residues, 70 or more contiguous amino acid residues, 80 or more contiguous amino acid residues, 90 or more contiguous amino acid residues, 100 or more contiguous amino acid residues, 125 or more contiguous amino acid residues, 150 or more contiguous amino acid residues, 175 or more contiguous amino acid residues, 200 or more contiguous amino acid residues, or 250 or more contiguous amino acid residues. The term “antigen-binding fragment”, in particular, includes Fab, F(ab′), F(ab′)2, Fv, dAb, Fd, complementarity determining region (CDR) fragments, single-chain antibodies (scFvs), bivalent single-chain antibodies, single-chain phage antibodies, unibodies, diabodies, triabodies, tetrabodies, polypeptides containing at least one fragment of immunoglobulin which is sufficient for a particular antigen to bind to the polypeptide, and the like. The fragment may be produced synthetically or by enzymatic or chemical digestion of a complete immunoglobulin, or may be produced by genetic engineering methods using recombinant DNA techniques. Production methods are well known in the art.

In the present invention, the Lrig-1 protein is a transmembrane protein consisting of 1091 amino acids present on the surface of regulatory T cells, and is composed of leucine-rich repeats (LRRs) and three immunoglobulin-like domains on the extracellular or lumen side, a cell transmembrane sequence, and a cytoplasmic tail portion. The LRIG gene family includes LRIG1, LRIG2, and LRIG3, and the amino acids therebetween are highly conserved. The LRIG1 gene is highly expressed in normal skin and can be expressed in basal and hair follicle cells to regulate proliferation of epithelial stem cells. Therefore, the LRIG1 gene plays an important role in maintaining homeostasis of the epidermis, and its absence may develop psoriasis or skin cancer. It has been reported that in a case where chromosome 3p14.3 portion in which LRIG1 is located is cut off, there is a possibility of developing into cancer cells. In fact, it was identified that expression of LRIG1 is greatly decreased in renal cell carcinoma and cutaneous squamous cell carcinoma. Recently, it has been also found that Lrig-1 is expressed in only about 20 to 30% of cancers. On the other hand, for the purpose of the present invention, the Lrig-1 protein may be, but is not limited to, a protein present in humans or mice.

In the present invention, the Lrig-1 protein may be, but is not limited to, a human-derived polypeptide represented by SEQ ID NO: 1 or a mouse-derived polypeptide represented by SEQ ID NO: 3.

In addition, in the present invention, the Lrig-1 protein represented by SEQ ID NO: 1 may be encoded by a polynucleotide represented by SEQ ID NO: 2, but is not limited thereto.

In addition, in the present invention, the Lrig-1 protein represented by SEQ ID NO: 3 may be encoded by a polynucleotide represented by SEQ ID NO: 4, but is not limited thereto.

In the present invention, the binding molecule may be a binding molecule, comprising:

a heavy chain variable region that contains a heavy chain CDR1 consisting of an amino acid sequence selected from the group consisting of SEQ ID NOs: 5, 13, 21, and 29; a heavy chain CDR2 consisting of an amino acid sequence selected from the group consisting of SEQ ID NOs: 6, 14, 22, and 30; a heavy chain CDR3 consisting of an amino acid sequence selected from the group consisting of SEQ ID NOs: 7, 15, 23, and 31; and

a light chain variable region that contains a light chain CDR1 consisting of an amino acid sequence selected from the group consisting of SEQ ID NOs: 8, 16, 24, and 32; a light chain CDR2 consisting of an amino acid sequence selected from the group consisting of SEQ ID NOs: 9, 17, 25, and 33; a light chain CDR3 consisting of an amino acid sequence selected from the group consisting of SEQ ID NO: 10, 18, 26, and 34.

In the present invention, the binding molecule may be a binding molecule, comprising:

a heavy chain variable region, selected from the group consisting of the following (a) to (d):

(a) a heavy chain variable region that contains a heavy chain CDR1 represented by SEQ ID NO: 5, a heavy chain CDR2 represented by SEQ ID NO: 6, and a heavy chain CDR3 represented by SEQ ID NO: 7;

(b) a heavy chain variable region that contains a heavy chain CDR1 represented by SEQ ID NO: 13, a heavy chain CDR2 represented by SEQ ID NO: 14, and a heavy chain CDR3 represented by SEQ ID NO: 15;

(c) a heavy chain variable region that contains a heavy chain CDR1 represented by SEQ ID NO: 21, a heavy chain CDR2 represented by SEQ ID NO: 22, and a heavy chain CDR3 represented by SEQ ID NO: 23; and

(d) a heavy chain variable region that contains a heavy chain CDR1 represented by SEQ ID NO: 29, a heavy chain CDR2 represented by SEQ ID NO: 30, and a heavy chain CDR3 represented by SEQ ID NO: 31; and

a light chain variable region, selected from the group consisting of the following (e) to (h):

(e) a light chain variable region that contains a light chain CDR1 represented by SEQ ID NO: 8, a light chain CDR2 represented by SEQ ID NO: 9, and a light chain CDR3 represented by SEQ ID NO: 10;

(f) a light chain variable region that contains a light chain CDR1 represented by SEQ ID NO: 16, a light chain CDR2 represented by SEQ ID NO: 17, and a light chain CDR3 represented by SEQ ID NO: 18;

(g) a light chain variable region that contains a light chain CDR1 represented by SEQ ID NO: 24, a light chain CDR2 represented by SEQ ID NO: 25, and a light chain CDR3 represented by SEQ ID NO: 26;

(h) a light chain variable region that contains a light chain CDR1 represented by SEQ ID NO: 32, a light chain CDR2 represented by SEQ ID NO: 33, and a light chain CDR3 represented by SEQ ID NO: 34.

In the present invention, the binding molecule may be a binding molecule selected from the group consisting of the following (1) to (4):

(1) a binding molecule comprising a heavy chain variable region that contains a heavy chain CDR1 represented by SEQ ID NO: 5, a heavy chain CDR2 represented by SEQ ID NO: 6, and a heavy chain CDR3 represented by SEQ ID NO: 7; and a light chain variable region that contains a light chain CDR1 represented by SEQ ID NO: 8, a light chain CDR2 represented by SEQ ID NO: 9, and a light chain CDR3 represented by SEQ ID NO: 10;

(2) a binding molecule comprising a heavy chain variable region that contains a heavy chain CDR1 represented by SEQ ID NO: 13, a heavy chain CDR2 represented by SEQ ID NO: 14, and a heavy chain CDR3 represented by SEQ ID NO: 15; and a light chain variable region that contains a light chain CDR1 represented by SEQ ID NO: 16, a light chain CDR2 represented by SEQ ID NO: 17, and a light chain CDR3 represented by SEQ ID NO: 18;

(3) a binding molecule comprising a heavy chain variable region that contains a heavy chain CDR1 represented by SEQ ID NO: 21, a heavy chain CDR2 represented by SEQ ID NO: 22, and a heavy chain CDR3 represented by SEQ ID NO: 23; and a light chain variable region that contains a light chain CDR1 represented by SEQ ID NO: 24, a light chain CDR2 represented by SEQ ID NO: 25, and a light chain CDR3 represented by SEQ ID NO: 26;

(4) a binding molecule comprising a heavy chain variable region that contains a heavy chain CDR1 represented by SEQ ID NO: 29, a heavy chain CDR2 represented by SEQ ID NO: 30, and a heavy chain CDR3 represented by SEQ ID NO: 31; and a light chain variable region that contains a light chain CDR1 represented by SEQ ID NO: 32, a light chain CDR2 represented by SEQ ID NO: 33, and a light chain CDR3 represented by SEQ ID NO: 34.

In the present invention, the binding molecule may be a binding molecule, comprising:

a heavy chain variable region consisting of any one amino acid sequence selected from the group consisting of SEQ ID NOs: 11, 19, 27, and 35; and

a light chain variable region consisting of any one amino acid sequence selected from the group consisting of SEQ ID NO: 12, 20, 28, and 36.

In the present invention, the binding molecule may be a binding molecule selected from the group consisting of the following binding molecules:

(i) a binding molecule comprising a heavy chain variable region represented by SEQ ID NO: 11, and a light chain variable region represented by SEQ ID NO: 12;

(ii) a binding molecule comprising a heavy chain variable region represented by SEQ ID NO: 19, and a light chain variable region represented by SEQ ID NO: 20;

(iii) a binding molecule comprising a heavy chain variable region represented by SEQ ID NO: 27, and a light chain variable region represented by SEQ ID NO: 28; and

(iv) a binding molecule comprising a heavy chain variable region represented by SEQ ID NO: 35, and a light chain variable region represented by SEQ ID NO: 36.

In the present invention, the binding molecule may further comprise a fragment crystallization (Fc) region or a constant region. Here, the Fc region may be an Fc region of an IgG1, IgG2, IgG3, or IgG4 antibody, or may be derived therefrom. Alternatively, the Fc region may be a hybrid Fc region.

In the present invention, the Fc region may be an Fc region of a mammalian-derived IgG1, IgG2, IgG3, or IgG4 antibody, and may preferably be an Fc region of a human-derived IgG1, IgG2, IgG3, or IgG4 antibody. However, the present invention is not limited thereto.

As an example of the present invention, the constant region may be a mouse-derived IgG2a constant region represented by SEQ ID NO: 37, but is not limited thereto.

As an example of the present invention, the constant region may be a mouse-derived immunoglobulin kappa constant region represented by SEQ ID NO: 38, but is not limited thereto.

As an example of the present invention, the constant region may be a human-derived IgG1 constant region represented by SEQ ID NO: 39 or 53, but is not limited thereto.

As an example of the present invention, the constant region may be a human-derived immunoglobulin kappa constant region represented by SEQ ID NO: 40, but is not limited thereto.

As an example of the present invention, the constant region may be a human-derived IgG2 constant region represented by SEQ ID NO: 41, but is not limited thereto.

As an example of the present invention, the constant region may be a human-derived IgG3 constant region represented by SEQ ID NO: 42, but is not limited thereto.

As an example of the present invention, the constant region may be a human-derived IgG4 constant region represented by SEQ ID NO: 43, but is not limited thereto.

As an example of the present invention, the Fc region may be a human-derived immunoglobulin lambda constant region, but is not limited thereto.

In the present invention, the “hybrid Fc” may be derived from a combination of human IgG subclasses or a combination of human IgD and IgG. In a case where the hybrid Fc binds to a biologically active molecule, polypeptide, or the like, the hybrid Fc has effects of not only increasing a serum half-life of the biologically active molecule, but also increasing an expression level of the polypeptide when a nucleotide sequence encoding the Fc-polypeptide fusion protein is expressed.

As an example of the present invention, the hybrid Fc region may be a hybrid Fc represented by SEQ ID NO: 44, but is not limited thereto.

In the binding molecule of the present invention, the Fc or constant region may be linked, via a linker, to the variable region. Here, the linker may be linked to the C-terminus of the Fc or constant region, and the N-terminus of the binding molecule of the present invention may be linked to the linker. However, the present invention is not limited thereto.

In the present invention, the “linker” may contain a sequence that can be cleaved by an enzyme that is overexpressed in a tissue or cell having a target disease. In a case where the linker may be cleaved by the overexpressed enzyme as described above, it is possible to effectively prevent activity of a polypeptide from decreasing due to the Fc or constant region. In the present invention, an example of the linker may be preferably a peptide linker consisting of 33 amino acids located in the 282^{nd} to 314^{th} portion of human albumin which is most abundantly present in the blood, and more preferably a peptide linker consisting of 13 amino acids located in the 292^{nd} to 304^{th} portion of the human albumin. Such portions are portions which are mostly exposed to the outside in three-dimensional structure, and thus have a minimum possibility of inducing an immune response in the body. However, the linker is not limited thereto.

The binding molecule of the present invention may further comprise a heavy chain constant region consisting of an amino acid sequence selected from the group consisting of SEQ ID NOs: 37, 39, 41, 42, 43, 44, and 53.

The binding molecule of the present invention may further comprise a light chain constant region consisting of an amino acid sequence represented by SEQ ID NO: 38 or 40.

The binding molecule of the present invention may further comprise:

a heavy chain constant region consisting of an amino acid sequence represented by SEQ ID NO: 37; and

a light chain constant region consisting of an amino acid sequence represented by SEQ ID NO: 38.

The binding molecule of the present invention may further comprise:

a heavy chain constant region consisting of an amino acid sequence represented by SEQ ID NO: 39, 41, 42, 43, and 53; and

a light chain constant region consisting of an amino acid sequence represented by SEQ ID NO: 40.

The binding molecule of the present invention may further comprise:

a heavy chain constant region consisting of an amino acid sequence represented by SEQ ID NO: 44.

The binding molecule of the present invention may be a binding molecule selected from the group of the following binding molecules:

a binding molecule comprising a heavy chain represented by SEQ ID NO: 45, and a light chain represented by SEQ ID NO: 46;

a binding molecule comprising a heavy chain represented by SEQ ID NO: 47, and a light chain represented by SEQ ID NO: 48;

a binding molecule comprising a heavy chain represented by SEQ ID NO: 49, and a light chain represented by SEQ ID NO: 50; and

a binding molecule comprising a heavy chain represented by SEQ ID NO: 51, and a light chain represented by SEQ ID NO: 52. The binding molecule of the present invention is characterized by being an antibody, but is not limited thereto. The antibody includes all of a monoclonal antibody, a full-length antibody, or an antibody fragment which is a portion of an antibody, has the ability to bind to Lrig-1 protein, and can compete with the binding molecule of the present invention in binding to an epitope on Lrig-1.

As used herein, the term “antibody” refers to a protein molecule which serves as a receptor that specifically recognizes an antigen, including an immunoglobulin molecule that is immunologically reactive with a particular antigen. For the purpose of the present invention, the antigen may be Lrig-1 protein present on the surface of regulatory T cells. Preferably, the antibody may specifically recognize the leucine-rich region or immunoglobulin-like domain of the Lrig-1 protein, but is not limited thereto.

In the present invention, the “immunoglobulin” has a heavy chain and a light chain, and each of the heavy chain and the light chain comprises a constant region and a variable region. The variable region of each of the light chain and the heavy chain contains three hypervariable regions called complementarity determining regions (hereinafter referred to as “CDRs”) and four framework regions. The CDRs primarily serve to bind to an epitope on an antigen. The CDRs of each chain are typically referred to as CDR1, CDR2, and CDR3 sequentially starting from the N-terminus, and are also distinguished by the chain where particular CDRs are located.

In addition, as used herein, the term “monoclonal antibody” refers to an antibody molecule of a single molecular composition which is obtained from substantially the same antibody population, and exhibits single binding specificity and affinity for a particular epitope.

In the present invention, the “full-length antibody” has a structure with two full-length light chains and two full-length heavy chains in which each light chain is linked to a heavy chain by disulfide bond, and includes IgA, IgD, IgE, IgM, and IgG. The IgG includes, as subtypes thereof, IgG1, IgG2, IgG3, and IgG4.

In addition, as used herein, the term “antigen fragment ” refers to a fragment that retains an antigen-binding function, and includes Fab, Fab′, F(ab′)₂, Fv, and the like. The Fab has a structure with variable regions of light and heavy chains, a constant region of the light chain, and a first constant region (CH1 domain) of the heavy chain, and has one antigen-binding site. In addition, Fab′ is different from Fab in that Fab′ has a hinge region containing at least one cysteine residue at the C-terminus of the heavy chain CH1 domain. F(ab′)₂ antibodies are produced with cysteine residues at the hinge region of Fab′ forming disulfide bond. Fv (variable fragment) refers to the smallest antibody fragment having only a heavy chain variable region and a light chain variable region. Double-chain Fv (dsFv) is configured to be such that a heavy chain variable region and a light chain variable region are linked to each other by disulfide bond, and single-chain Fv (scFv) is configured to be such that a heavy chain variable region and a light chain variable region are covalently linked to each other, in general, via a peptide linker. The antibody fragment may be obtained as Fab or F(ab′)₂ fragment in a case where a proteolytic enzyme, for example, papain or pepsin is used, and may be produced through a genetic recombinant technique.

In addition, in the present invention, the antibody may be, but is not limited to, a chimeric antibody, a humanized antibody, a bivalent, bispecific molecule, a minibody, a domain antibody, a bispecific antibody, an antibody mimetic, a unibody, a diabody, a triabody, or a tetrabody, or a fragment thereof.

In the present invention, the “chimeric antibody” is an antibody which is obtained by recombination of a variable region of a mouse antibody and a constant region of a human antibody, and has a greatly improved immune response as compared with the mouse antibody.

In addition, as used herein, the term “humanized antibody” refers to an antibody obtained by modifying a protein sequence of an antibody derived from a non-human species so that the protein sequence is similar to an antibody variant naturally produced in humans. For example, the humanized antibody may be prepared as follows. Mouse-derived CDRs may be recombined with a human antibody-derived FR to prepare a humanized variable region, and the humanized variable region may be recombined with a constant region of a preferred human antibody to prepare a humanized antibody.

In the present invention, the binding molecule may be provided as a bispecific antibody or a bispecific antigen-binding fragment which is capable of binding to Lrig-1 protein and also binding to another protein.

In the present invention, the bispecific antibody and the bispecific antigen-binding fragment may comprise the binding molecule according to the present invention. As an example of the present invention, the bispecific antibody and the bispecific antigen-binding fragment comprise an antigen-binding domain capable of binding to Lrig-1 protein, wherein the antigen-binding domain capable of binding to Lrig-1 protein may comprise or consist of the binding molecule according to the present invention.

The bispecific antibody and the bispecific antigen-binding fragment provided in the present invention comprise an antigen-binding domain, which is a binding molecule capable of binding to Lrig-1 protein according to the present invention, and an antigen-binding domain capable of binding to another target protein. Here, the antigen-binding domain capable of binding another target protein may be an antigen-binding domain capable of binding to a protein other than Lrig-1 protein, for example, PD-1 or a cell surface receptor. However, the antigen-binding domain is not limited thereto.

The bispecific antibody and the bispecific antigen-binding fragment according to the present invention may be provided in any suitable format, for example, that described in Kontermann MAbs 2012, 4(2): 182-197, which is incorporated herein by reference in its entirety. For example, the bispecific antibody or the bispecific antigen-binding fragment may be a bispecific antibody conjugate (for example, IgG2, F(ab′)2, or CovX-body), a bispecific IgG or IgG-like molecule (for example, IgG, scFv4-Ig, IgG-scFv, scFv-IgG, DVD-Ig, IgG-sVD, sVD-IgG, or 2 in 1-IgG, mAb2, or Tandemab common LC), an asymmetric bispecific IgG or IgG-like molecule (for example, kih IgG, kih IgG common LC, CrossMab, kih IgG-scFab, mAb-Fv, charge pair, or SEED-body), a small bispecific antibody molecule (for example, diabody (Db), dsDb, DART, scDb, tandAb, tandem scFv (taFv), tandem dAb/VHH, triple body, triple head, Fab-scFv, or F(ab′)2-scFv2), a bispecific Fc and CH3 fusion protein (for example, taFv-Fc, di-diabody, scDb-CH3, scFv-Fc-scFv, HCAb-VHH, scFv-kih-Fc, or scFv-kih-CH3), or a bispecific fusion protein (for example, scFv2-albumin, scDb-albumin, taFv-toxin, DNL-Fab3, DNL-Fab4-IgG, DNL-Fab4-IgG-cytokine 2). See, in particular, FIG. 2 in Kontermann MAbs 2012, 4(2): 182-19. The bispecific antibody and the bispecific antigen-binding fragment according to the invention may be designed and prepared by those skilled in the art.

A method for producing the bispecific antibody in the present invention comprises forming a reducing disulfide or non-reducing thioether bond, and chemical crosslinking of an antibody or antibody fragment as described, for example, in Segal and Bast, 2001. Production of Bispecific Antibodies. Current Protocols in Immunology. 14:IV:2.13:2.13.1-2.13.16, which is incorporated herein by reference in its entirety. For example, N-succinimidyl-3-(-2-pyridyldithio)-propionate (SPDP) may be used, for example, for chemically crosslinking an Fab fragment through an SH-group at the hinge region, to generate a disulfide-linked bispecific F(ab)2 heterodimer.

In addition, an alternative method for producing the bispecific antibody in the present invention comprises fusing an antibody-producing hybridoma with, for example, polyethylene glycol, to produce quadroma cells capable of secreting bispecific antibodies, as described, for example, in D. M. and Bast, B. J. 2001. Production of Bispecific Antibodies. Current Protocols in Immunology. 14:IV:2.13:2.13.1-2.13.16.

The bispecific antibody and the bispecific antigen-binding fragment according to the invention may also be, for example, recombinantly produced by expression from a nucleic acid construct that encodes a polypeptide for an antigen-binding molecule, as described, for example, in Antibody Engineering: Methods and Protocols, Second Edition (Humana Press, 2012), at Chapter 40: Production of Bispecific Antibodies: Diabodies and Tandem scFv (Hornig and Farber-Schwarz), or French, How to make bispecific antibodies, Methods Mol. Med. 2000; 40:333-339, both of which are incorporated herein by reference in their entireties.

For example, a DNA construct that contains a sequence encoding light and heavy chain variable domains for two antigen-binding domains (that is, light and heavy chain variable domains for an antigen-binding domain capable of binding to PD-1 or the like, and light and heavy chain variable domains for an antigen-binding domain capable of binding to another target protein), and a sequence encoding a suitable linker or dimerization domain between the antigen-binding domains may be prepared by molecular cloning techniques. Subsequently, a recombinant bispecific antibody may be produced by expression of the construct (for example, in vitro) in a suitable host cell (for example, a mammalian host cell), and then the expressed recombinant bispecific antibody may be optionally purified.

Antibodies may be produced by an affinity maturation process in which a modified antibody with improved affinity for an antigen as compared with an unmodified parent antibody is produced. An affinity matured antibody may be produced by a procedure known in the art, for example, in Marks et al., Rio/Technology 10:779-783 (1992); Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-159 (1995); and Hawkins et al, J. Mol. Biol. 226:889-896 (1992).

In addition, the binding molecule provided in the present invention may include a variant of the amino acid sequence as long as the variant can specifically bind to Lrig-1 protein. For example, in order to improve binding affinity and/or other biological properties of an antibody, modifications may be made to an amino acid sequence of the antibody. Such modifications include, for example, deletions, insertions, and/or substitutions of amino acid sequence residues of the antibody.

Such amino acid variations are made based on relative similarity of amino acid side chain substituents such as hydrophobicity, hydrophilicity, charge, and size. According to analysis on sizes, shapes, and types of amino acid side chain substituents, it can be seen that arginine, lysine, and histidine are all positively charged residues; alanine, glycine, and serine have similar sizes; and phenylalanine, tryptophan, and tyrosine have similar shapes. Thus, based on these considerations, it can be said that arginine, lysine, and histidine; alanine, glycine, and serine; and phenylalanine, tryptophan, and tyrosine are biologically functional equivalents.

In introducing variations, the hydropathic index of amino acids may be considered. Each amino acid has been assigned hydropathic index depending on its hydrophobicity and charge: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The hydropathic amino acid index is very important in conferring the interactive biological function on a protein. It is known that substitution with an amino acid having similar hydropathic index allows a protein to retain similar biological activity. In a case where variations are introduced with reference to the hydropathic index, substitutions are made between amino acids that exhibit a hydropathic index difference of preferably within ±2, more preferably within ±1, and even more preferably within ±0.5.

Meanwhile, it is also well known that substitutions between amino acids having similar hydrophilicity values result in proteins with equivalent biological activity. As disclosed in U.S. Pat. No. 4,554,101, respective amino acid residues have been assigned the following hydrophilicity values: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In a case where variations are introduced with reference to the hydrophilicity values, substitutions may be made between amino acids that exhibit a hydrophilicity value difference of preferably within ±2, more preferably within ±1, and even more preferably within ±0.5.

Amino acid exchanges in proteins which do not entirely alter activity of a molecule are known in the art (H. Neurath, R. L. Hill, The Proteins, Academic Press, New York (1979)). The most commonly occurring exchanges are exchanges between amino acid residues Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Gln/Glu.

Given the above-described variations with biologically equivalent activity, it is interpreted that the binding molecule of the present invention also includes sequences that exhibit substantial identity with the sequences listed in the Sequence Listing.

As used herein, the term “substantial identity” refers to a sequence showing at least 61% homology, more preferably 70% homology, even more preferably 80% homology, and most preferably 90% homology when the sequence of the present invention is aligned with any other sequence so that they maximally correspond to each other, and the aligned sequence is analyzed by using an algorithm typically used in the art. Alignment methods for comparison of sequences are known in the art. Various methods and algorithms for alignment are disclosed in Smith and Waterman, Adv. Appl. Math. 2:482(1981); Needleman and Wunsch, J. Mol. Bio. 48:443(1970); Pearson and Lipman, Methods in Mol. Biol. 24: 307-31(1988); Higgins and Sharp, Gene 73:237-44(1988); Higgins and Sharp, CABIOS 5:151-3(1989); Corpet et al., Nuc. Acids Res. 16:10881-90(1988); Huang et al., Comp. Appl. BioSci. 8:155-65(1992); and Pearson et al., Meth. Mol. Biol. 24:307-31(1994). NCBI Basic Local Alignment Search Tool (BLAST) (Altschul et al., J. Mol. Biol. 215: 403-10 (1990)) is accessible from the National Center for Biological Information (NBCI), or the like, and may be used in conjunction with sequencing programs, such as blastp, blasm, blastx, tblastn, and tblastx, on the internet. BLSAT is accessible at http://www.ncbi.nlm.nih.gov/BLAST/. Sequence homology comparison methods using this program can be identified online (http://www.ncbi.nlm.nih.gov/BLAST/blast_help.html).

In the present invention, the binding molecule, preferably the antibody, may be produced by a conventional method for producing an antibody, and may be produced by affinity maturation.

As used herein, the term “affinity maturation” refers to a process in which antibodies having increased affinity for an antigen are produced by activated B cells in the course of an immune response. For the purpose of the present invention, the affinity maturation allows antibodies or antibody fragments to be produced due to affinity maturation based on the principles of mutation and selection, in the same process that occurs in nature.

The binding molecule, preferably the antibody, provided in the present invention can activate the function, particularly of regulatory T immune cells (Treg cells), among immune cells; increase the number of the Treg cells; and regulate immunological tolerance, thereby effectively preventing, ameliorating, or treating an immune-related disease.

In the present invention, the “immune-related disease” may be a disease induced by excessive activation and expression of various immune cells and inflammatory cells. The immune-related disease may, for example, include autoimmune diseases; graft-versus-host diseases; organ transplant rejection; asthma; atopy; or acute or chronic inflammatory diseases, but is not limited thereto.

In addition, in the present invention, the “autoimmune disease” may be, but is not limited to, one or more selected from the group consisting of rheumatoid arthritis, systemic scleroderma, systemic lupus erythematosus, atopic dermatitis, psoriasis, alopecia areata, asthma, Crohn's disease, Behcet's disease, Sjogren's syndrome, Guillain—Barré syndrome, chronic thyroiditis, multiple sclerosis, multiple myositis, ankylosing spondylitis, fibrositis, and polyarteritis nodosa.

According to another embodiment of the present invention, there is provided a nucleic acid molecule encoding the binding molecule provided in the present invention.

The nucleic acid molecule of the present invention includes all nucleic acid molecules obtained by translating the amino acid sequences of the binding molecules provided in the present invention to polynucleotide sequences, as known to those skilled in the art. Therefore, various polynucleotide sequences may be prepared by an open reading frame (ORF), and all of these polynucleotide sequences are also included in the nucleic acid molecule of the present invention.

According to yet another embodiment of the present invention, there is provided an expression vector into which the isolated nucleic acid molecule provided in the present invention is inserted.

In the present invention, the “vector” is a nucleic acid molecule capable of transporting another nucleic acid linked thereto. One type of vector is a “plasmid,” which refers to circular double-stranded DNA into which an additional DNA segment can be ligated. Another type of vector is a phage vector. Yet another type of vector is a viral vector, where an additional DNA segment can be ligated into the viral genome. Certain vectors are capable of autonomous replication in a host cell into which they are introduced (for example, bacterial vectors having a bacterial origin of replication and episomal mammalian vectors). Other vectors (for example, non-episomal mammalian vectors) can be integrated into the genome of a host cell upon introduction into the host cell, and thus are replicated along with the host genome. In addition, certain vectors are capable of directing expression of genes to which they are operatively linked. Such vectors are referred to herein as “recombinant expression vectors” or simply “expression vectors.” In general, expression vectors useful in recombinant DNA techniques are often in the form of plasmids. In the present specification, “plasmid” and “vector” may be used interchangeably as the plasmid is the most commonly used form of vector.

Specific examples of the expression vector in the present invention may be selected from, but are not limited to, the group consisting of commercially widely used pCDNA vectors, F, R1, RP1, Col, pBR322, ToL, Ti vectors; cosmids; phages such as lambda, lambdoid, M13, Mu, p1 P22, Qμμ, T-even, T2, T3, T7; plant viruses. Any expression vector known, to those skilled in the art, as expression vectors can be used in the present invention, and the expression vector is selected depending on the nature of the target host cell. Introduction of a vector into a host cell may be performed by calcium phosphate transfection, viral infection, DEAE-dextran-mediated transfection, lipofectamine transfection, or electroporation. However, the present invention is not limited thereto, and those skilled in the art may adopt and use an introduction method appropriate for the expression vector and the host cell which are used. The vector may preferably contain at least one selection marker. However, the present invention is not limited thereto, and selection can be made using the vector that contains no selection marker, depending on whether or not a product is produced. The selection marker is selected depending on the target host cell, which is done using methods already known to those skilled in the art, and thus the present invention has no limitation thereon.

In the present invention, to facilitate purification of the nucleic acid molecule, a tag sequence may be inserted into and fused to an expression vector. The tag includes, but is not limited to, hexa-histidine tag, hemagglutinin tag, myc tag, or flag tag, and any tag known to those skilled in the art which facilitates purification can be used in the present invention.

According to still yet another embodiment of the present invention, there is provided a host cell line transfected with the expression vector provided in the present invention.

In the present invention, the “host cell” includes individual cells or cell cultures which may be or have been recipients of the vector(s) for incorporation of a polypeptide insert. The host cell includes progeny of a single host cell, and the progeny may not necessarily be completely identical (in morphology or in genomic DNA complement) to the original parent cell due to natural, accidental, or intentional mutation. The host cell includes cells transfected in vivo with the polynucleotide(s) herein.

In the present invention, the host cell may include cells of mammalian, plant, insect, fungal, or cellular origin, and may be, for example, bacterial cells such as E. coli, Streptomyces, Salmonella typhimurium; fungal cells such as yeast cells and Pichia pastoris; insect cells such as Drosophila and Spodoptera Sf9 cells; animal cells such as Chinese hamster ovary (CHO) cells, SP2/0 (mouse myeloma), human lymphoblastoid, COS, NSO (mouse myeloma), 293T, Bowes melanoma cells, HT-1080, baby hamster kidney (BHK) cells, human embryonic kidney (HEK) cells, or PERC.6 (human retinal cells); or plant cells. However, the host cell is not limited thereto, and any cell known to those skilled in the art which can be used as a host cell line is available.

In the present invention, the transfection method may be any method of injecting a desired vector into the host cell, and include any known method capable of injecting a vector into a host cell. Examples of the transfection method may include, but are not limited to, CaCl₂-mediated transfection, electroporation, microinjection, calcium phosphate precipitation, electroporation, liposome-mediated transfection, DEAE-dextran treatment, gene bombardment, and virus-mediated transfection.

According to still yet another embodiment of the present invention, there is provided an antibody-drug conjugate (ADC) comprising the antibody provided in the present invention and a drug.

As used herein, the term “antibody-drug conjugate (ADC)” refers to a form in which the drug and the antibody are chemically linked to each other without degrading biological activity of the antibody and the drug. In the present invention, the antibody-drug conjugate denotes a form in which the drug is bound to an amino acid residue at the N-terminus of the heavy and/or light chain of the antibody, specifically, a form in which the drug is bound to an α-amine group at the N-terminus of the heavy and/or light chain of the antibody.

As used herein, the term “drug” may mean any substance having a certain biological activity for a cell, which is a concept including DNA, RNA, or a peptide. The drug may be in a form which contains a reactive group capable of reacting and crosslinking with an α-amine group, and also includes a form which contains a reactive group capable of reacting and crosslinking with an α-amine group and to which a linker is linked.

In the present invention, examples of the reactive group capable of reacting and crosslinking with the α-amine group are not particularly limited in terms of type as long as the reactive group can react and crosslink with an α-amine group at the N-terminus of a heavy or light chain of an antibody. The reactive group includes all types of groups known in the art which react with an amine group. The reactive group may, for example, be any one of isothiocyanate, isocyanate, acyl azide, NHS ester, sulfonyl chloride, aldehyde, glyoxal, epoxide, oxirane, carbonate, aryl halide, imidoester, carbodiimide, anhydride, and fluorophenyl ester, but is not limited thereto.

In the present invention, the drug includes any drug regardless of type as long as the drug can treat diseases targeted by the Lrig-1 antibody, and may preferably include therapeutic agents for immune-related diseases, for example, autoimmune diseases, graft versus host diseases, organ transplant rejection, asthma, atopy, acute or chronic inflammatory diseases, or the like.

According to still yet another embodiment of the present invention, there is provided a pharmaceutical composition for preventing or treating an immune-related disease, comprising, as an active ingredient, the binding molecule or antibody-drug conjugate (ADC) provided in the present invention.

The binding molecule, preferably the antibody, provided in the present invention can activate the function, particularly of regulatory T immune cells (Treg cells), among immune cells; increase the number of the Treg cells; and regulate immunological tolerance, thereby effectively preventing, ameliorating, or treating an immune-related disease.

In the present invention, the “immune-related disease” may be a disease induced by excessive activation and expression of various immune cells and inflammatory cells. The immune-related disease may, for example, include autoimmune diseases; graft-versus-host diseases; organ transplant rejection; asthma; atopy; or acute or chronic inflammatory diseases, but is not limited thereto.

In addition, in the present invention, the “autoimmune disease” may be, but is not limited to, one or more selected from the group consisting of rheumatoid arthritis, systemic scleroderma, systemic lupus erythematosus, atopic dermatitis, psoriasis, alopecia areata, asthma, Crohn's disease, Behcet's disease, Sjogren's syndrome, Guillain—Barré syndrome, chronic thyroiditis, multiple sclerosis, multiple myositis, ankylosing spondylitis, fibrositis, and polyarteritis nodosa.

On the other hand, in the present invention, the “prevention” may include, without limitation, any act of blocking symptoms of a disease, or suppressing or delaying the symptoms, using the pharmaceutical composition of the present invention.

In addition, in the present invention, the “treatment” may include, without limitation, any act of ameliorating or beneficially altering symptoms of a disease, using the pharmaceutical composition of the present invention.

In the present invention, the pharmaceutical composition may be characterized by being in the form of capsules, tablets, granules, injections, ointments, powders, or beverages, and the pharmaceutical composition may be characterized by being targeted to humans.

In the present invention, the pharmaceutical composition may be formulated in the form of oral preparations such as powders, granules, capsules, tablets, and aqueous suspensions, preparations for external use, suppositories, and sterile injectable solutions, respectively, according to conventional methods, and used. However, the pharmaceutical composition is not limited thereto. The pharmaceutical composition of the present invention may further comprise a pharmaceutically acceptable carrier. As the pharmaceutically acceptable carrier, a binder, a glidant, a disintegrant, an excipient, a solubilizer, a dispersant, a stabilizer, a suspending agent, a pigment, a flavor, and the like may be used for oral administration; a buffer, a preserving agent, a pain-relieving agent, a solubilizer, an isotonic agent, a stabilizer, and the like may be used in admixture for injections; and a base, an excipient, a lubricant, a preserving agent, and the like may be used for topical administration. The preparations of the pharmaceutical composition of the present invention may be prepared in various ways by being mixed with the pharmaceutically acceptable carrier as described above. For example, for oral administration, the pharmaceutical composition may be formulated in the form of tablets, troches, capsules, elixirs, suspensions, syrups, wafers, or the like. For injections, the pharmaceutical composition may be formulated in the form of unit dosage ampoules or multiple dosage forms. Alternatively, the pharmaceutical composition may be formulated into solutions, suspensions, tablets, capsules, sustained-release preparations, or the like.

Meanwhile, as examples of carriers, diluents, or excipients suitable for making preparations, lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, mineral oil, or the like may be used. In addition, a filler, an anti-coagulant, a lubricant, a wetting agent, a fragrance, an emulsifier, a preservative, and the like may further be included.

The route of administration of the pharmaceutical composition of the present invention includes, but is not limited to, oral, intravenous, intramuscular, intraarterial, intramedullary, intradural, intracardiac, transdermal, subcutaneous, intraperitoneal, intranasal, intestinal, topical, sublingual, or rectal route. Oral or parenteral administration is preferred.

In the present invention, the “parenteral” includes subcutaneous, intradermal, intravenous, intramuscular, intraarticular, intrabursal, intrasternal, intradural, intralesional, and intracranial injection or infusion techniques. The pharmaceutical composition of the present invention may also be administered in the form of suppositories for rectal administration.

The pharmaceutical composition of the present invention may vary depending on a variety of factors, including activity of a certain compound used, the patient's age, body weight, general health status, sex, diet, time of administration, route of administration, rate of excretion, drug combination, and severity of a certain disease to be prevented or treated. A dose of the pharmaceutical composition may vary depending on the patient's condition, body weight, severity of disease, drug form, route of administration, and duration, and may be appropriately selected by those skilled in the art. The pharmaceutical composition may be administered in an amount of 0.0001 to 50 mg/kg or 0.001 to 50 mg/kg, per day. Administration may be made once a day or several times a day. The dose is not intended to limit the scope of the invention in any case. The pharmaceutical composition according to the present invention may be formulated in the form of pills, sugar-coated tablets, capsules, liquids, gels, syrups, slurries, or suspensions.

According to still yet another embodiment of the present invention, there is provided a method for preventing, ameliorating, or treating an immune-related disease, comprising a step of administering, to an individual, a pharmaceutically effective amount of the binding molecule or antibody-drug conjugate (ADC) provided in the present invention.

The binding molecule, preferably the antibody, provided in the present invention can activate the function, particularly of regulatory T immune cells (Treg cells), among immune cells; increase the number of the Treg cells; and regulate immunological tolerance, thereby effectively preventing, ameliorating, or treating an immune-related disease.

In the present invention, the “individual” is an individual suspected of developing an immune-related disease, and the individual suspected of developing cancer or an immune-related disease means a mammal, such as humans, mice, and domestic animals, who has developed or is likely to develop the disease in question. However, any individual, who is treatable with the binding molecule or antibody-drug conjugate provided in the present invention is included therein without limitation.

The method of the present invention may comprise administering a pharmaceutically effective amount of the binding molecule or antibody-drug conjugate provided in the present invention. An appropriate total daily amount used may be determined by an attending physician or veterinarian within the scope of sound medical judgment, and administration may be made once or several times. However, for the purposes of the present invention, a specific therapeutically effective amount for a particular patient is preferably applied differently depending on various factors, including type and degree of reaction to be achieved, a composition comprising the above-mentioned specific active ingredient, including whether other agents are used therewith as the case may be, the patient's age, body weight, general health status, sex, and diet, time of administration, route of administration, secretion rate of the composition comprising the above-mentioned specific active ingredient, duration of treatment, and drugs used simultaneously or in combination with the specific composition, and similar factors well known in the medical field.

Meanwhile, the method for preventing or treating an immune-related disease may be, but is not limited to, a combination therapy that further comprises administering a compound or substance having therapeutic activity against one or more diseases.

In the present invention, the “combination” should be understood to represent simultaneous, individual, or sequential administration. In a case where the administration is made in a sequential or individual manner, the second component should be administered at intervals such that beneficial effects of the combination are not lost.

In the present invention, the dosage of the binding molecule or antibody-drug conjugate (ADC) may be, but is not limited to, about 0.0001 μg to 500 mg per kg of patient's body weight.

### Advantageous Effects of Invention

The binding molecule, preferably the antibody, specific for the Lrig-1 protein according to the present invention can activate the function, particularly of regulatory T immune cells (Treg cells), among immune cells; increase the number of the Treg cells; and regulate immunological tolerance, thereby effectively preventing, ameliorating, or treating diseases induced by excessive activation and expression of various immune cells and inflammatory cells, for example, immune-related diseases such as autoimmune diseases; graft-versus-host diseases; organ transplant rejection; asthma; atopy; or acute or chronic inflammatory diseases.

In addition, the binding molecule, preferably the antibody, specific for the Lrig-1 protein according to the present invention has advantages of more effectively targeting the Lrig-1 protein as compared with antibodies against Lrig-1 which are previously commercially available, and also possessing very good binding capacity thereto.

### BRIEF DESCRIPTION OF DRAWINGS

FIG. 1 illustrates a structure of the Lrig-1 protein according to an embodiment of the present invention.

FIG. 2 illustrates a structure of the Lrig-1 protein according to an embodiment of the present invention.

FIG. 3 illustrates prediction results for epitopes of the Lrig-1 protein according to an embodiment of the present invention.

FIG. 4 illustrates prediction results for epitopes of the Lrig-1 protein according to an embodiment of the present invention.

FIG. 5 illustrates an expression level of Lrig-1 mRNA according to an embodiment of the present invention.

FIG. 6 illustrates an expression level of Lrig-1 mRNA according to an embodiment of the present invention.

FIG. 7 illustrates an expression level of Lrig-1 mRNA according to an embodiment of the present invention.

FIG. 8 illustrates expression levels of Lrig-1, Lrig-2, and Lrig-3 mRNAs according to an embodiment of the present invention.

FIG. 9 illustrates results obtained by comparing expression levels of Lrig-1 protein in regulatory T cells and non-regulated T cells according to an embodiment of the present invention.

FIG. 10 illustrates expression of the Lrig-1 protein on the surface of regulatory T cells according to an embodiment of the present invention.

FIG. 11 illustrates results obtained by analyzing binding capacity of Lrig-1 protein-specific monoclonal antibodies (A7, C8, E7, and G3) to the Lrig-1 protein according to an embodiment of the present invention.

FIG. 12 illustrates results obtained by analyzing the mechanism of regulating Lrig-1 protein-induced Stat3 phosphorylation, in regulatory T cells, of Lrig-1 protein-specific monoclonal antibodies (A7, C8, E7, and G3) according to an embodiment of the present invention.

FIG. 13 illustrates results obtained by analyzing therapeutic effects, on an autoimmune disease, of Lrig-1 protein-specific monoclonal antibodies (A7, C8, E7, and G3) according to an embodiment of the present invention.

### DETAILED DESCRIPTION OF INVENTION

According to an embodiment of the present invention, there is provided a binding molecule selected from the group consisting of the following (1) to (4):

(1) a binding molecule comprising a heavy chain variable region that contains a heavy chain CDR1 represented by SEQ ID NO: 5, a heavy chain CDR2 represented by SEQ ID NO: 6, and a heavy chain CDR3 represented by SEQ ID NO: 7; and a light chain variable region that contains a light chain CDR1 represented by SEQ ID NO: 8, a light chain CDR2 represented by SEQ ID NO: 9, and a light chain CDR3 represented by SEQ ID NO: 10;

(2) a binding molecule comprising a heavy chain variable region that contains a heavy chain CDR1 represented by SEQ ID NO: 13, a heavy chain CDR2 represented by SEQ ID NO: 14, and a heavy chain CDR3 represented by SEQ ID NO: 15; and a light chain variable region that contains a light chain CDR1 represented by SEQ ID NO: 16, a light chain CDR2 represented by SEQ ID NO: 17, and a light chain CDR3 represented by SEQ ID NO: 18;

(3) a binding molecule comprising a heavy chain variable region that contains a heavy chain CDR1 represented by SEQ ID NO: 21, a heavy chain CDR2 represented by SEQ ID NO: 22, and a heavy chain CDR3 represented by SEQ ID NO: 23; and a light chain variable region that contains a light chain CDR1 represented by SEQ ID NO: 24, a light chain CDR2 represented by SEQ ID NO: 25, and a light chain CDR3 represented by SEQ ID NO: 26;

(4) a binding molecule comprising a heavy chain variable region that contains a heavy chain CDR1 represented by SEQ ID NO: 29, a heavy chain CDR2 represented by SEQ ID NO: 30, and a heavy chain CDR3 represented by SEQ ID NO: 31; and a light chain variable region that contains a light chain CDR1 represented by SEQ ID NO: 32, a light chain CDR2 represented by SEQ ID NO: 33, and a light chain CDR3 represented by SEQ ID NO: 34.

Hereinafter, the present invention will be described in more detail by way of examples. These examples are only for describing the present invention in more detail, and it will be apparent to those skilled in the art that according to the gist of the present invention, the scope of the present invention is not limited by these examples.

### EXAMPLES

### [Preparation Example 1] T Cell Subset Cell Culture

In order to identify whether the Lrig-1 protein is expressed only in regulatory T cells (Treg), the subsets of T cells, Th0, Th1, Th2, Th17, and iTreg, were prepared. The iTreg refers to cells whose differentiation has been artificially induced in a medium containing the following composition, unlike nTreg which has been naturally isolated.

The subsets of the T cells were induced to differentiate into respective cells by first isolating naive T cells obtained from the spleen of mice, causing RPMI1640 (Invitrogen Gibco, Grand Island, N.Y.) nutrient medium that contains 10% fetal bovine serum (FBS; HyClone, Logan, Utah) to further contain the respective ingredients of Table 1 below, and performing 72-hour incubation in an incubator at 37° C., 5% CO₂.

TABLE 1

| Differentiated cell | Composition |
|---|---|
| Th0 | anti-CD3, anti-CD28 |
| Th1 | IL-12, anti-IL-4 antibody |
| Th2 | IL-4, anti-IFNβ |
| Th17 | IL-6, TGFβ, anti-IFNβ, anti-IL-4 |
| iTreg | IL-2, TGFβ |

### [Example 1] Structural Analysis of Lrig-1

A three-dimensional steric structure of the extracellular domain of the Lrig-1 protein was predicted to produce antibodies specific for the Lrig-1 protein, a surface protein of regulatory T cells.

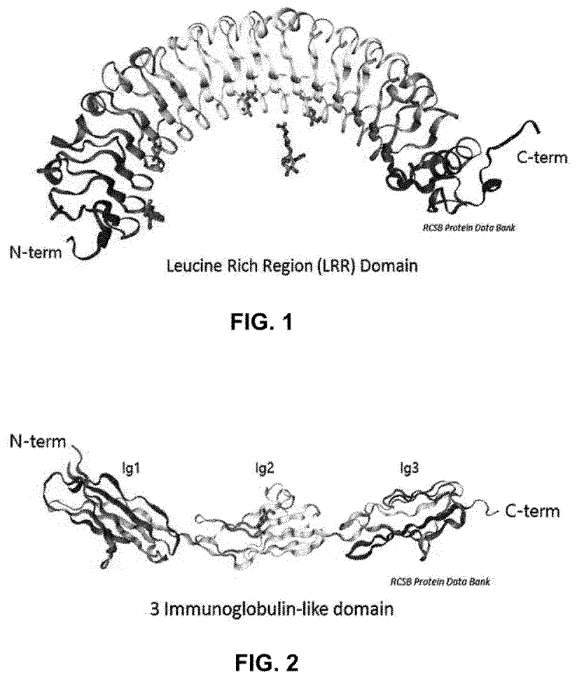

First, in order to predict base sequences of epitopes (epitopes), tools of Uniprot (http://www.uniprot.org) and RCSB Protein Data Bank (http://www.rcsb.org/pdb) were used to predict a three-dimensional steric structure of the extracellular domain (ECD) of the Lrig-1 protein so that the structure of ECD is identified. Then, the results are illustrated in FIGS. 1 and 2.

As illustrated in FIG. 1, a total of 15 leucine-rich regions of LRR1 to LRR15 existed in the Lrig-LRR domain (amino acid sequence at positions 41 to 494) in the extracellular domain of the Lrig-1 protein. Each of the LRR domains is composed of 23 to 27 amino acids, with 3 to 5 leucine being present.

In addition, as illustrated in FIG. 2, three immunoglobulin-like domains exist in amino acid sequences at positions 494 to 781 of the Lrig-1 protein in the extracellular domain of the Lrig-1 protein.

### [Example 2] Prediction of Lrig-1 Epitope Amino Acid Sequence

Prediction of the above base sequence was performed using Ellipro server (http://tools.iedb.org/ellipro/) which is an epitope prediction software based on a structure of the Lrig-1 protein. The Ellipro search engine was used because it corresponds to a search engine known to be the most reliable among the existing algorithms for predicting an epitope.

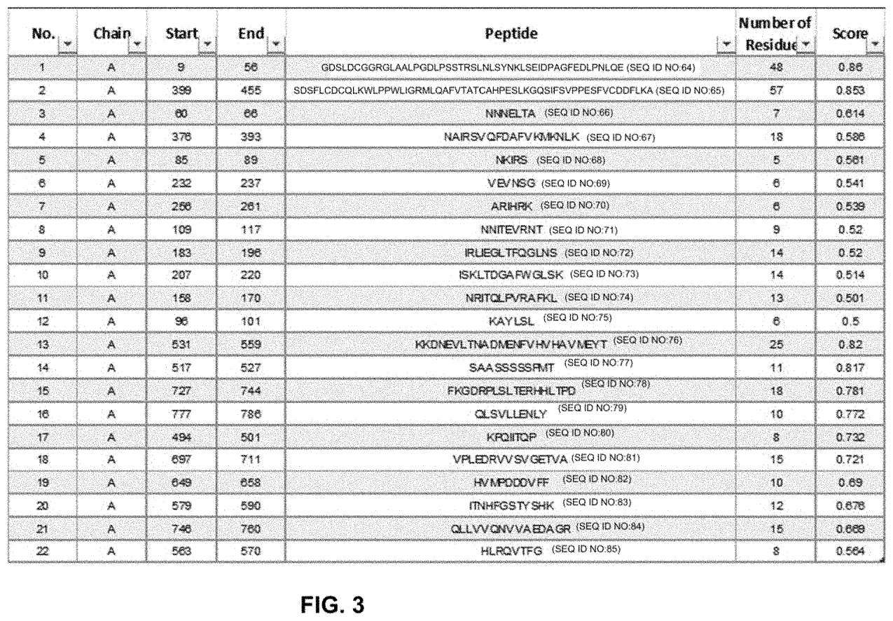

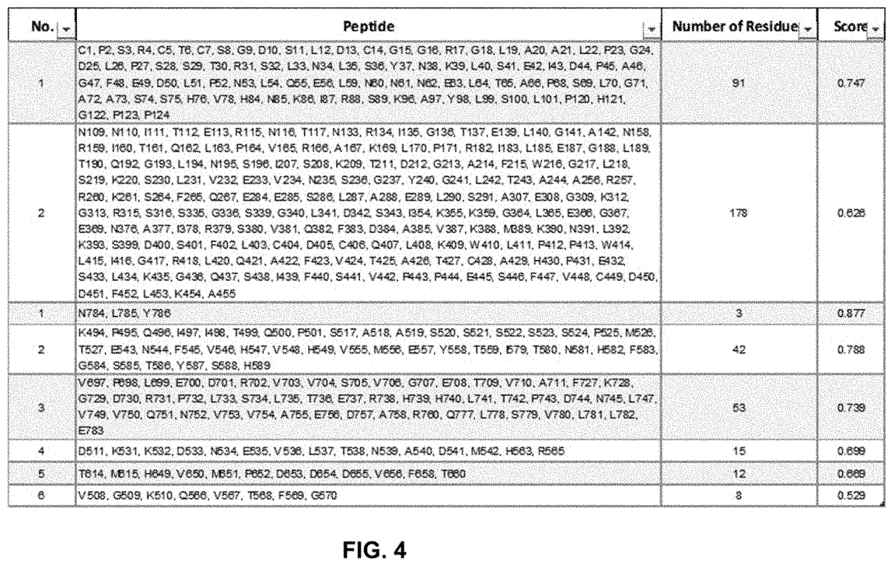

The extracellular domain analyzed in Example 1 was entered into the epitope prediction software, and then predicted contiguous or discontiguous amino acid sequences of the predicted epitopes are illustrated in FIGS. 3 and 4.

As illustrated in FIGS. 3 and 4, a total of 22 contiguous epitope amino acid sequences were predicted, and a total of 8 discontiguous epitope amino acid sequences were predicted.

### [Production Examples 1 to 4] Production of Monoclonal Antibodies Specific to Lrig-1 Protein

Antibodies specific for the Lrig-1 protein according to the present invention were produced. The present antibodies were not produced by specifying a certain epitope, but were produced as antibodies capable of binding to any site on the Lrig-1 protein.

In order to produce the antibodies, cells expressing the Lrig-1 protein were produced. More specifically, a DNA fragment corresponding to SEQ ID NO: 2 and pcDNA (hygro) were cleaved with a cleavage enzyme, incubated at 37° C., and ligated to produce pcDNA into which a DNA sequence of the Lrig-1 protein is inserted. The thus produced pcDNA into which SEQ ID NO: 2 is inserted was introduced, through transfection, into L cells, so that the Lrig-1 protein is allowed to be expressed on the surface of the L cells.

Light and heavy chain amino acid sequences capable of binding to Lrig-1 expressed on the cell surface were selected from the Human scFv library so that a total of eight heavy and light chains were selected.

The selected heavy and light chain amino acid sequences were fused with the mlgG2a Fc region, to produce monoclonal antibodies. The sequences of the monoclonal antibodies are shown in Table 2 below.

TABLE 2

| | | | | Sequence |
|---|---|---|---|---|
| Classification | Clone | Location | Amino acid sequence | information |
| Production | A7 | Heavy | EVQLLESGGGLVQPGGSLRLSCAASGFTFSGYD | SEQ ID NO: 45 |
| Example 1 | clone | chain | MSWVRQAPGKGLEWVSLIYPDSGNKYYADSVK | |
| | | | GRFTISRDNSKNTLYLQMNSLRAEDTAVYYCAR | |
| | | | DAGLSWAGAFDYWGQGTLVTVSSTTAPSVYPL | |
| | | | APVCGDTTGSSVTLGCLVKGYFPEPVTLTWNSG | |
| | | | SLSSGVHTFPAVLQSDLYTLSSSVTVTSSTWPSQS | |
| | | | ITCNVAHPASSTKVDKKIEPRGPTIKPCPPCKCPA | |
| | | | PNLLGGPSVFIFPPKIKDVLMISLSPIVTCVVVDVS | |
| | | | EDDPDVQISWFVNNVEVHTAQTQTHREDYNSTL | |
| | | | RVVSALPIQHQDWMSGKEFKCKVNNKDLPAPIE | |
| | | | RTISKPKGSVRAPQVYVLPPPEEEMTKKQVTLTC | |
| | | | MVTDFMPEDIYVEWTNNGKTELNYKNTEPVLDS | |
| | | | DGSYFMYSKLRVEKKNWVERNSYSCSVVHEGL | |
| | | | HNHHTTKSFSRTPGK | |
| | | Light | QSVLTQPPSASGTPGQRVTISCSGSSSNIGSNYVT | SEQ ID NO: 46 |
| | | chain | WYQQLPGTAPKLLIYSDSHRPSGVPDRFSGSKSG | |
| | | | TSASLAISGLQSEDEADYYCGSWDYSLSAYVFG | |
| | | | GGTKLTVLRTVAAPTVSIFPPSSEQLTSGGASVV | |
| | | | CFLNNFYPKDINVKWKIDGSERQNGVLNSWTDQ | |
| | | | DSKDSTYSMSSTLTLTKDEYERHNSYTCEATHKT | |
| | | | STSPIVKSFNRNEC | |
| Production | C8 | Heavy | EVQLLESGGGLVQPGGSLRLSCAASGFTFSNYY | SEQ ID NO: 47 |
| Example 2 | clone | chain | MSWVRQAPGKGLEWVSGISPGDSSTYYADSVK | |
| | | | GRFTISRDNSKNTLYLQMNSLRAEDTAVYYCAK | |
| | | | GLYSNPNEPFDYWGQGTLVTVSSTTAPSVYPLAP | |
| | | | VCGDTTGSSVTLGCLVKGYFPEPVTLTWNSGSLS | |
| | | | SGVHTFPAVLQSDLYTLSSSVTVTSSTWPSQSITC | |
| | | | NVAHPASSTKVDKKIEPRGPTIKPCPPCKCPAPNL | |
| | | | LGGPSVFIFPPKIKDVLMISLSPIVTCVVVDVSED | |
| | | | DPDVQISWFVNNVEVHTAQTQTHREDYNSTLRV | |
| | | | VSALPIQHQDWMSGKEFKCKVNNKDLPAPIERTI | |
| | | | SKPKGSVRAPQVYVLPPPEEEMTKKQVTLTCMV | |
| | | | TDFMPEDIYVEWTNNGKTELNYKNTEPVLDSDG | |
| | | | SYFMYSKLRVEKKNWVERNSYSCSVVHEGLHN | |
| | | | HHTTKSFSRTPGK | |
| | | Light | QSVLTQPPSASGTPGQRVTISCTGSSSNIGSNYVS | SEQ ID NO: 48 |
| | | chain | WYQQLPGTAPKLLIYDDSQRPSGVPDRFSGSKSG | |
| | | | TSASLAISGLRSEDEADYYCGTWDYSLNGYVFG | |
| | | | GGTKLTVLRTVAAPTVSIFPPSSEQLTSGGASVV | |
| | | | CFLNNFYPKDINVKWKIDGSERQNGVLNSWTDQ | |
| | | | DSKDSTYSMSSTLTLTKDEYERHNSYTCEATHKT | |
| | | | STSPIVKSFNRNEC | |
| Production | E7 | Heavy | EVQLLESGGGLVQPGGSLRLSCAASGFTFSSYDM | SEQ ID NO: 49 |
| Example 3 | clone | chain | SWVRQAPGKGLEWVSGISPDGSNIYYADSVKGR | |
| | | | FTISRDNSKNTLYLQMNSLRAEDTAVYYCAKVG | |
| | | | LRCRYEACSYAYGMDVWGQGTLVTVSSTTAPS | |
| | | | VYPLAPVCGDTTGSSVTLGCLVKGYFPEPVTLT | |
| | | | WNSGSLSSGVHTFPAVLQSDLYTLSSSVTVTSST | |
| | | | WPSQSITCNVAHPASSTKVDKKIEPRGPTIKPCPP | |
| | | | CKCPAPNLLGGPSVFIFPPKIKDVLMISLSPIVTCV | |
| | | | VVDVSEDDPDVQISWFVNNVEVHTAQTQTHRED | |
| | | | YNSTLRVVSALPIQHQDWMSGKEFKCKVNNKD | |
| | | | LPAPIERTISKPKGSVRAPQVYVLPPPEEEMTKKQ | |
| | | | VTLTCMVTDFMPEDIYVEWTNNGKTELNYKNTE | |
| | | | PVLDSDGSYFMYSKLRVEKKNWVERNSYSCSV | |
| | | | VHEGLHNHHTTKSFSRTPGK | |
| | | Light | QSVLTQPPSASGTPGQRVTISCSGSSSNIGSNYVS | SEQ ID NO: 50 |
| | | chain | WYQQLPGTAPKLLIYSDSHRPSGVPDRFSGSKSG | |
| | | | TSASLAISGLRSEDEADYYCATWDSSLNGYVFG | |
| | | | GGTKLTVLRTVAAPTVSIFPPSSEQLTSGGASVV | |
| | | | CFLNNFYPKDINVKWKIDGSERQNGVLNSWTDQ | |
| | | | DSKDSTYSMSSTLTLTKDEYERHNSYTCEATHKT | |
| | | | STSPIVKSFNRNEC | |
| Production | G3 | Heavy | EVQLLESGGGLVQPGGSLRLSCAASGFTFSNYD | SEQ ID NO: 51 |
| Example 4 | clone | chain | MSWVRQAPGKGLEWVSSISPSSGSIYYADSVKG | |
| | | | RFTISRDNSKNTLYLQMNSLRAEDTAVYYCAKD | |
| | | | LDAFWRPSFDYWGQGTLVTVSSTTAPSVYPLAP | |
| | | | VCGDTTGSSVTLGCLVKGYFPEPVTLTWNSGSLS | |
| | | | SGVHTFPAVLQSDLYTLSSSVTVTSSTWPSQSITC | |
| | | | NVAHPASSTKVDKKIEPRGPTIKPCPPCKCPAPNL | |
| | | | LGGPSVFIFPPKIKDVLMISLSPIVTCVVVDVSED | |
| | | | DPDVQISWFVNNVEVHTAQTQTHREDYNSTLRV | |
| | | | VSALPIQHQDWMSGKEFKCKVNNKDLPAPIERTI | |
| | | | SKPKGSVRAPQVYVLPPPEEEMTKKQVTLTCMV | |
| | | | TDFMPEDIYVEWTNNGKTELNYKNTEPVLDSDG | |
| | | | SYFMYSKLRVEKKNWVERNSYSCSVVHEGLHN | |
| | | | HHTTKSFSRTPGK | |
| | | Light | QSVLTQPPSASGTPGQRVTISCTGSSSNIGNNNVN | SEQ ID NO: 52 |
| | | chain | WYQQLPGTAPKLLIYSDSHRPSGVPDRFSGSKSG | |
| | | | TSASLAISGLRSEDEADYYCGSWDDSLSAYVFGG | |
| | | | GTKLTVLRTVAAPTVSIFPPSSEQLTSGGASVVCF | |
| | | | LNNFYPKDINVKWKIDGSERQNGVLNSWTDQDS | |
| | | | KDSTYSMSSTLTLTKDEYERHNSYTCEATHKTST | |
| | | | SPIVKSFNRNEC | |
| Production | A8 | Heavy | EVQLLESGGGLVQPGGSLRLSCAASGFTFSDYD | — |
| Example 5 | clone | chain | MSWVRQVPGKGLEWVSWISHGGGSIYYADSVK | |
| | | | GRFTISRDNSKNTLYLQMNSLRAEDTAVYYCAR | |
| | | | GLGLCKTGLCYYYDAMDVWGQGTLVTVSSTTA | |
| | | | PSVYPLAPVCGDTTGSSVTLGCLVKGYFPEPVTL | |
| | | | TWNSGSLSSGVHTFPAVLQSDLYTLSSSVTVTSS | |
| | | | TWPSQSITCNVAHPASSTKVDKKIEPRGPTIKPCP | |
| | | | PCKCPAPNLLGGPSVFIFPPKIKDVLMISLSPIVTC | |
| | | | VVVDVSEDDPDVQISWFVNNVEVHTAQTQTHRE | |
| | | | DYNSTLRVVSALPIQHQDWMSGKEFKCKVNNK | |
| | | | DLPAPIERTISKPKGSVRAPQVYVLPPPEEEMTKK | |
| | | | QVTLTCMVTDFMPEDIYVEWTNNGKTELNYKN | |
| | | | TEPVLDSDGSYFMYSKLRVEKKNWVERNSYSCS | |
| | | | VVHEGLHNHHTTKSFSRTPGK | |
| | | Light | QSVLTQPPSASGTPGQRVTISCTGSSSNIGNNSVT | — |
| | | chain | WYQQLPGTAPKLLIYADNNRPSGVPDRFSGSKS | |
| | | | GTSASLAISGLRSEDEADYYCAAWDSSLSAYVFG | |
| | | | GGTKLTVLRTVAAPTVSIFPPSSEQLTSGGASVV | |
| | | | CFLNNFYPKDINVKWKIDGSERQNGVLNSWTDQ | |
| | | | DSKDSTYSMSSTLTLTKDEYERHNSYTCEATHKT | |
| | | | STSPIVKSFNRNEC | |
| Production | B8 | Heavy | EVQLLESGGGLVQPGGSLRLSCAASGFTFSDYY | — |
| Example 6 | clone | chain | MSWVRQAPGKGLEWVSGISHDSGSKYYADSVK | |
| | | | GRFTISRDNSKNTLYLQMNSLRAEDTAVYYCAR | |
| | | | HWTTFDYWGQGTLVTVSSTTAPSVYPLAPVCGD | |
| | | | TTGSSVTLGCLVKGYFPEPVTLTWNSGSLSSGVH | |
| | | | TFPAVLQSDLYTLSSSVTVTSSTWPSQSITCNVAH | |
| | | | PASSTKVDKKIEPRGPTIKPCPPCKCPAPNLLGGP | |
| | | | SVFIFPPKIKDVLMISLSPIVTCVVVDVSEDDPDV | |
| | | | QISWFVNNVEVHTAQTQTHREDYNSTLRVVSAL | |
| | | | PIQHQDWMSGKEFKCKVNNKDLPAPIERTISKPK | |
| | | | GSVRAPQVYVLPPPEEEMTKKQVTLTCMVTDFM | |
| | | | PEDIYVEWTNNGKTELNYKNTEPVLDSDGSYFM | |
| | | | YSKLRVEKKNWVERNSYSCSVVHEGLHNHHTT | |
| | | | KSFSRTPGK | |
| | | Light | QSVLTQPPSASGTPGQRVTISCSGSSSNIGSNNVT | — |
| | | chain | WYQQLPGTAPKLLIYANSNRPSGVPDRFSGSKSG | |
| | | | TSASLAISGLRSEDEADYYCGAWDYSLSAYVFG | |
| | | | GGTKLTVLRTVAAPTVSIFPPSSEQLTSGGASVV | |
| | | | CFLNNFYPKDINVKWKIDGSERQNGVLNSWTDQ | |
| | | | DSKDSTYSMSSTLTLTKDEYERHNSYTCEATHKT | |
| | | | STSPIVKSFNRNEC | |
| Production | D9 | Heavy | EVQLLESGGGLVQPGGSLRLSCAASGFTFSNYA | — |
| Example 7 | clone | chain | MSWVRQAPGKGLEWVSAIYPGGGSIYYADSVK | |
| | | | GRFTISRDNSKNTLYLQMNSLRAEDTAVYYCAR | |
| | | | DILPCPWGRCYYDYAMDVWGQGTLVTVSSTTA | |
| | | | PSVYPLAPVCGDTTGSSVTLGCLVKGYFPEPVTL | |
| | | | TWNSGSLSSGVHTFPAVLQSDLYTLSSSVTVTSS | |
| | | | TWPSQSITCNVAHPASSTKVDKKIEPRGPTIKPCP | |
| | | | PCKCPAPNLLGGPSVFIFPPKIKDVLMISLSPIVTC | |
| | | | VVVDVSEDDPDVQISWFVNNVEVHTAQTQTHRE | |
| | | | DYNSTLRVVSALPIQHQDWMSGKEFKCKVNNK | |
| | | | DLPAPIERTISKPKGSVRAPQVYVLPPPEEEMTKK | |
| | | | QVTLTCMVTDFMPEDIYVEWTNNGKTELNYKN | |
| | | | TEPVLDSDGSYFMYSKLRVEKKNWVERNSYSCS | |
| | | | VVHEGLHNHHTTKSFSRTPGK | |
| | | Light | QSVLTQPPSASGTPGQRVTISCSDSSSNIGSNTVS | — |
| | | chain | WYQQLPGTAPKLLIYADNNRPSGVPDRFSGSKS | |
| | | | GTSASLAISGLRSEDEADYYCGTWDYSLSGYVF | |
| | | | GGGTKLTVLRTVAAPTVSIFPPSSEQLTSGGASV | |
| | | | VCFLNNFYPKDINVKWKIDGSERQNGVLNSWTD | |
| | | | QDSKDSTYSMSSTLTLTKDEYERHNSYTCEATH | |
| | | | KTSTSPIVKSFNRNEC | |
| Production | H6 | Heavy | EVQLLESGGGLVQPGGSLRLSCAASGFTFSNYA | — |
| Example 8 | clone | chain | MSWVRQAPGKGLEWVSVISHGGGSTYYADSVK | |
| | | | GRFTISRDNSKNTLYLQMNSLRAEDTAVYYCAR | |
| | | | VISNCHLGVCYYSNGMDVWGQGTLVTVSSTTAP | |
| | | | SVYPLAPVCGDTTGSSVTLGCLVKGYFPEPVTLT | |
| | | | WNSGSLSSGVHTFPAVLQSDLYTLSSSVTVTSST | |
| | | | WPSQSITCNVAHPASSTKVDKKIEPRGPTIKPCPP | |
| | | | CKCPAPNLLGGPSVFIFPPKIKDVLMISLSPIVTCV | |
| | | | VVDVSEDDPDVQISWFVNNVEVHTAQTQTHRED | |
| | | | YNSTLRVVSALPIQHQDWMSGKEFKCKVNNKD | |
| | | | LPAPIERTISKPKGSVRAPQVYVLPPPEEEMTKKQ | |
| | | | VTLTCMVTDFMPEDIYVEWTNNGKTELNYKNTE | |
| | | | PVLDSDGSYFMYSKLRVEKKNWVERNSYSCSV | |
| | | | VHEGLHNHHTTKSFSRTPGK | |
| | | Light | QSVLTQPPSASGTPGQRVTISCSGSSSNIGNNDVY | — |
| | | chain | WYQQLPGTAPKLLIYSDSQRPSGVPDRFSGSKSG | |
| | | | TSASLAISGLRSEDEADYYCGTWDYSLSGYVFG | |
| | | | GGTKLTVLRTVAAPTVSIFPPSSEQLTSGGASVV | |
| | | | CFLNNFYPKDINVKWKIDGSERQNGVLNSWTDQ | |
| | | | DSKDSTYSMSSTLTLTKDEYERHNSYTCEATHKT | |
| | | | STSPIVKSFNRNEC | |

### [Example 3] Identification of Specific Expression of Lrig-1 mRNA in Regulatory T Cells

Verification was made of whether the Lrig-1 protein can act as a biomarker specific for regulatory T cells.

For the verification, CD4⁺ T cells were isolated using magnet-activated cell sorting (MACS), through CD4 beads, from the spleen of rats. Subsequently, regulatory T (CD4⁺CD25⁺ T) cells and non-regulatory T (CD4⁺CD25⁻ T) cells were isolated with a fluorescence-activated cell sorter (FACS) using a CD25 antibody. For the respective cells and the cells differentiated in Preparation Example 1, mRNA was extracted using Trizol, and gDNA was removed from genomic RNA using gDNA extraction kit (Qiagen) according to the protocol provided by the manufacturer. The gDNA-removed mRNA was synthesized into cDNA through the BDsprint cDNA Synthesis Kit (Clonetech).

Real-time polymerase chain reaction (RT PCR) was performed to quantitatively identify an expression level of Lrig-1 mRNA in the cDNA.

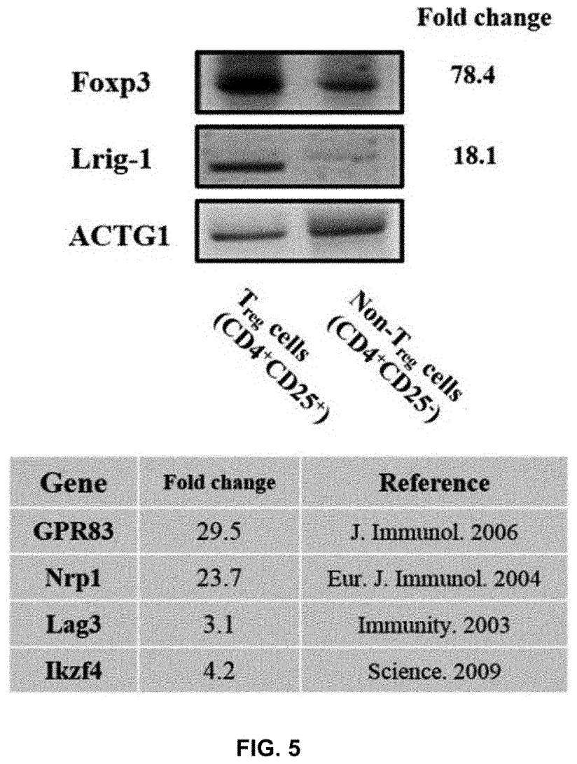

The real-time polymerase chain reaction was performed with primers shown in Table 3 below using SYBR Green (Molecular Probes) by the protocol provided by the manufacturer under conditions of 40 cycles consisting of 95° C. for 3 minutes, 61° C. for 15 seconds, 72° C. for 30 seconds, a relative gene expression level was calculated using the ΔΔCT method, and normalized using HPRT. The results are illustrated in FIGS. 5 to 8.

TABLE 3

| Primer | Sequence |
|---|---|
| Mouse Lrig-1 | Forward 5′-GAC GGA ATT CAG TGA GGA GAA CCT-3′ |
| | Reverse 5′-CAA CTG GTA GTG GCA GCT TGT AGG-3′ |
| Mouse Lrig-2 | forward 5′-TCA CAA GGA ACA TTG TCT GAA CCA-3′ |
| | reverse 5′-GCC TGA TCT AAC ACA TCC TCC TCA-3′ |
| Mouse Lrig-3 | forward 5′-CAG CAC CTT GAG CTG AAC AGA AAC-3′ |
| | reverse 5′-CCA GCC TTT GGT AAT CTC GGT TAG-3′ |
| Mouse FOXP3 | forward 5′-CTT TCA CCT ATC CCA CCC TTA TCC-3′ |
| | reverse 5′-ATT CAT CTA CGG TCC ACA CTG CTC-3′ |
| ACTG1 | forward 5′-GGC GTC ATG GTG GGC ATG GG-3′ |
| | reverse 5′-ATG GCG TGG GGA AGG GCG TA-3′ |

As illustrated in FIG. 5, it can be seen that the expression of Lrig-1 in regulatory T (CD4⁺CD25⁺ T) cells is 18.1 times higher than non-regulatory T (CD4⁺CD25⁻ T) cells. This was about 10 times higher expression level than Lag3 and Ikzf4, which are previously known markers for regulatory T cells.

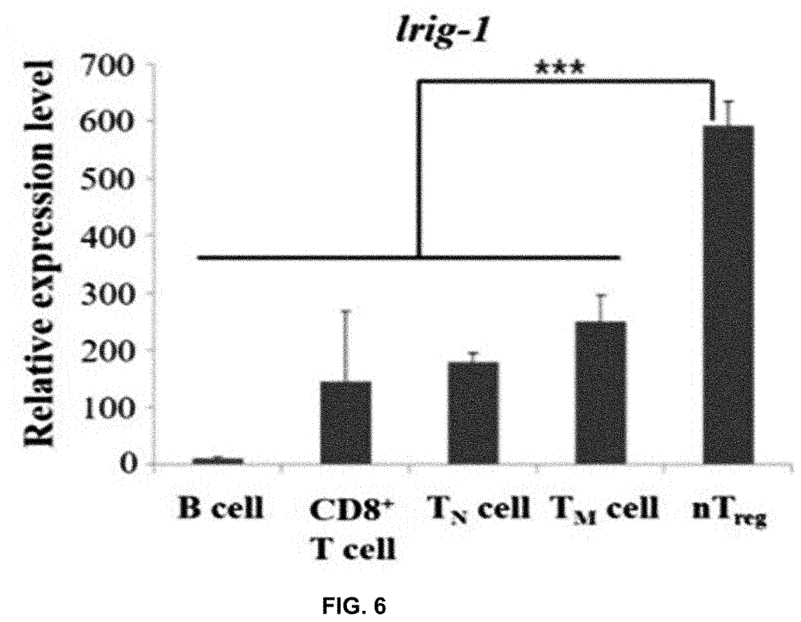

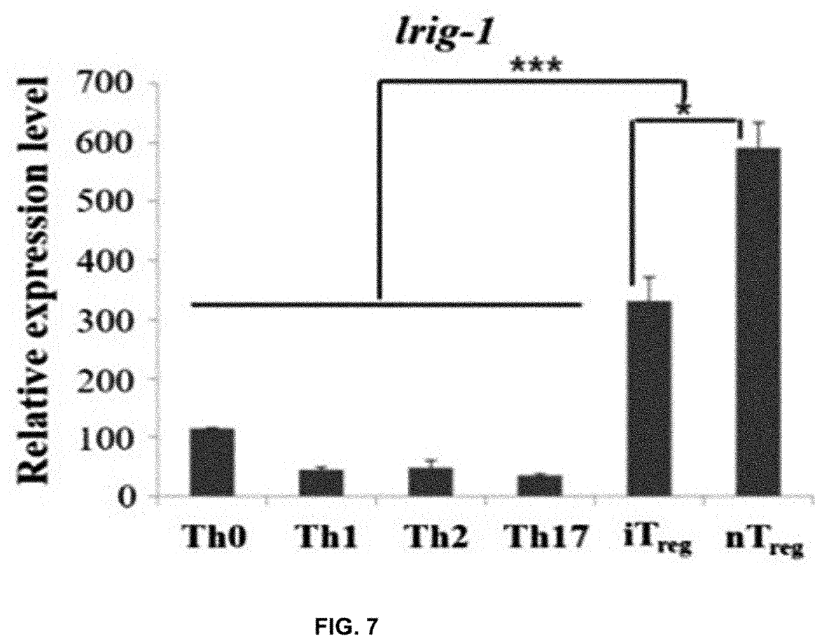

In addition, as illustrated in FIGS. 6 and 7, the expression of Lrig-1 mRNA was remarkably high in regulatory T cells as compared with other types of immune cells, and in particular, was remarkably high in naturally isolated regulatory T cells (nTreg) as compared with induced regulatory T cells (iTreg cells).

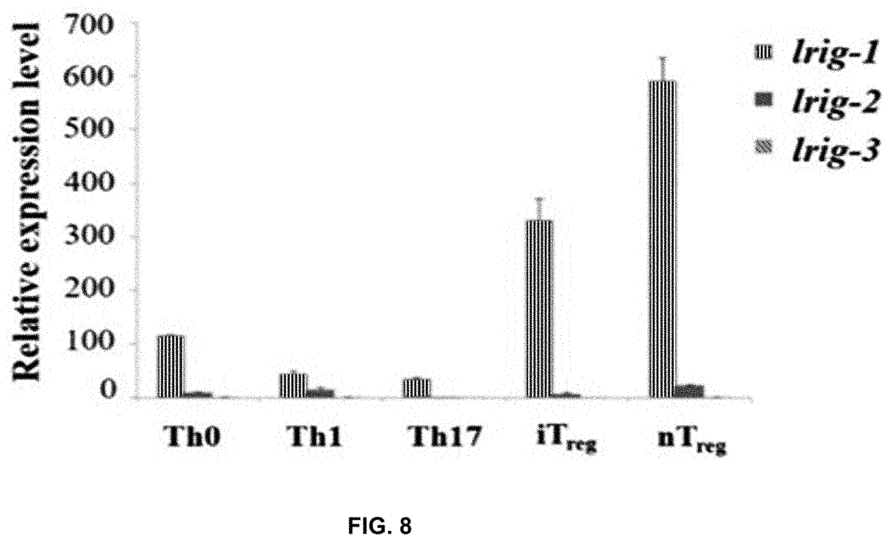

In addition, as illustrated in FIG. 8, expression of Lrig-1 was the highest among Lrig-1, Lrig-2, and Lrig-3 which correspond to the Lrig family.

From the above results, it can be seen that the Lrig-1 protein according to the present invention is specifically expressed in regulatory T cells, in particular, naturally-occurring regulatory T cells.

### [Example 4] Identification of Specific Expression of Lrig-1 Protein in Regulatory T Cells

It was identified whether the Lrig-1 protein expressed from Lrig-1 mRNA is specifically expressed only in regulatory T cells.

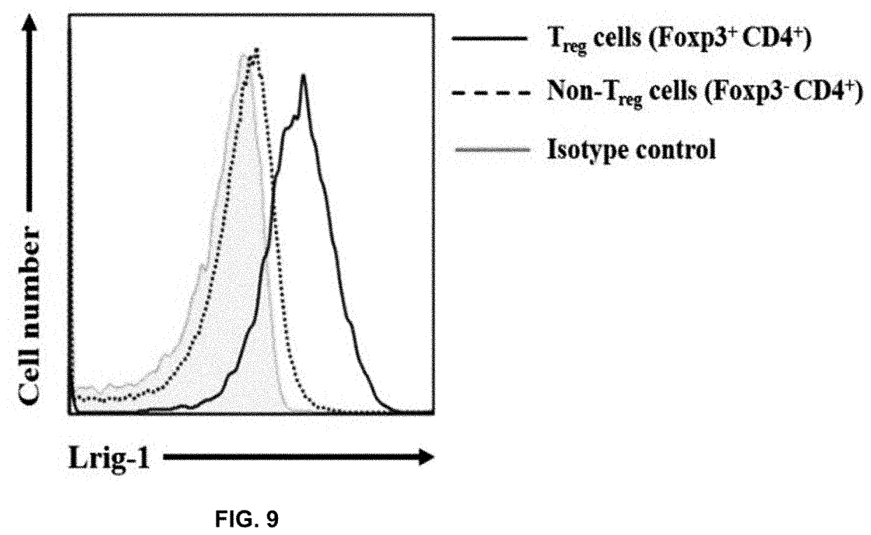

Using FOXP3-RFP-knocked-in mice, the FOXP3-RFP obtained by coupling red fluorescence protein (RFP) to FOXP3 promoter, a transcription factor specific for regulatory T cells, CD4⁺ T cells were isolated using magnet-activated cell sorting (MACS), through CD4 beads, from the spleen of the mice. Subsequently, using RFP protein, regulatory T (CD4⁺RFP⁺ T) cells and non-regulatory T (CD4⁺RFP⁻ T) cells were obtained by performing isolation through a fluorescence-activated cell sorter (FACS). The respective cells were stained with the purchased Lrig-1 antibody and a negative control was stained with an isotype-matched control antibody, to measure an expression level of Lrig-1 with the fluorescence-activated cell sorter. The results are illustrated in FIG. 9.

As illustrated in FIG. 9, the non-regulatory T cells indicated by a dotted line showed almost the same expression level of Lrig-1 as the negative control, whereas there were a large number of cells with high expression level of Lrig-1 in the regulatory T cells.

From the above results, it can be seen that the Lrig-1 protein according to the present invention is specifically expressed in regulatory T cells.

### [Example 5] Identification of Specific Expression of Lrig-1 Protein on Surface of Regulatory T Cells

From the viewpoint that in order to be a target of cell therapy, the Lrig-1 protein must be expressed on the surface of regulatory T cells, which in turn allows a more effective target therapy, it was identified whether the Lrig-1 protein is expressed on the surface of the regulatory T cells.

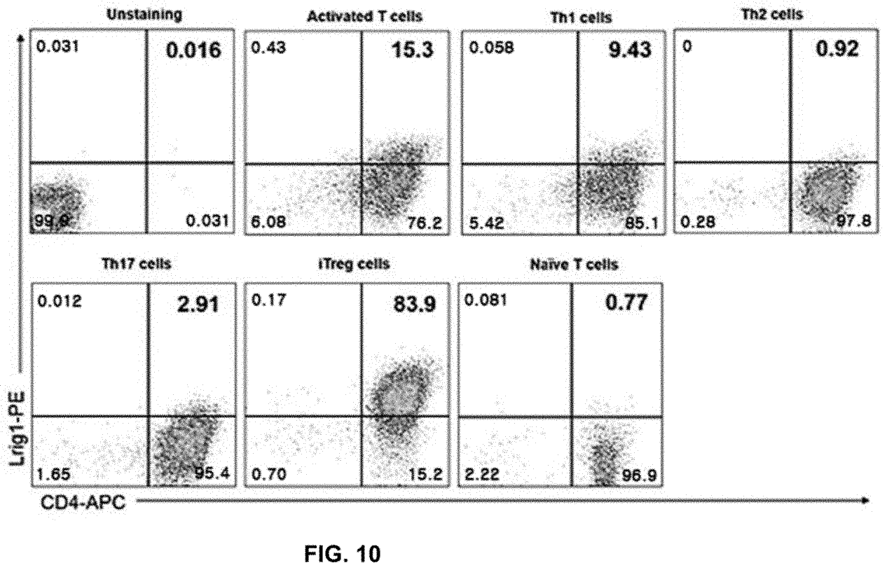

The respective differentiated T cell subsets of Preparation Example 1 were stained with anti-CD4-APC and anti-Lrig-1-PE antibodies, and expression levels of Lrig-1 were measured at the respective cell surfaces using a fluorescence-activated cell sorter (FACS). The results are illustrated in FIG. 10.

As illustrated in FIG. 10, Lrig-1 was expressed in an amount of 0.77 to 15.3 in activated T cells, Th1 cells, Th2 cells, Th17 cells, and naive T cells, whereas Lrig-1 was expressed as high as 83.9 in differentiation-induced T cells (iTreg cells).

From the above results, it can be seen that the Lrig-1 protein according to the present invention is not only specifically expressed in regulatory T (Treg) cells, but also is, in particular, expressed at a higher level on the surface of the Treg cells.

### [Example 6] Evaluation of Binding Capacity of Antibody According to Present Invention to Lrig-1 Protein

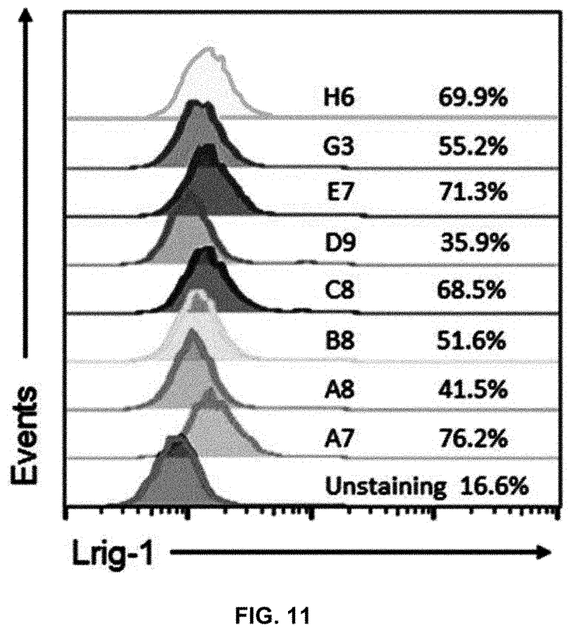

In order to identify whether the monoclonal antibodies according to the present invention produced in Production Examples 1 to 8 well recognize Lrig-1, each of the antibodies of Production Examples 1 to 8 was bound to L cells that stably express Lrig-1. Then, a secondary antibody which is conjugated with eFlour 670 and is capable of recognizing the mouse antibodies was added thereto, and then binding capacity of the monoclonal antibodies to the Lrig-1 protein was analyzed using FACS. The results are illustrated in FIG. 11.

As illustrated in FIG. 11, it was found that all Lrig-1 protein-specific monoclonal antibodies (A7, C8, E7, and G3) according to the present invention effectively recognize and bind to the Lrig-1 protein present on the surface of L cells.

### [Example 7] Regulation of Signal Transduction Pathway in Treg Cells, by Antibody According to Present Invention

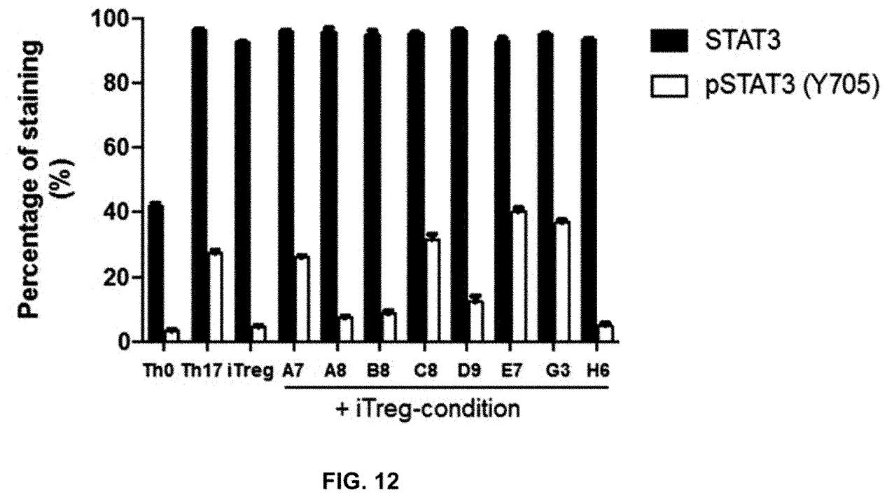

In order to analyze how the monoclonal antibodies according to the present invention produced in Production Examples 1 to 8 affect the signal transduction pathway in Treg cells through the Lrig-1 protein, Lrig-1 present on the surface of the Treg cells was stimulated by treating the Treg cells with the antibodies of Production Examples 1 to 8, and then a level of tyrosine phosphorylation of Stat3 protein present in the stimulated Treg cells was analyzed through phosphotyrosine immunoblot. The results are illustrated in FIG. 12.

As illustrated in FIG. 12, it was found that the Lrig-1 protein-specific monoclonal antibodies (A7, C8, E7, and G3) according to the present invention increase phosphorylation of Stat3 at the same level as Th17 cells.

### [Example 8] Therapeutic Effects of Antibody According to the Present Invention on Autoimmune Disease

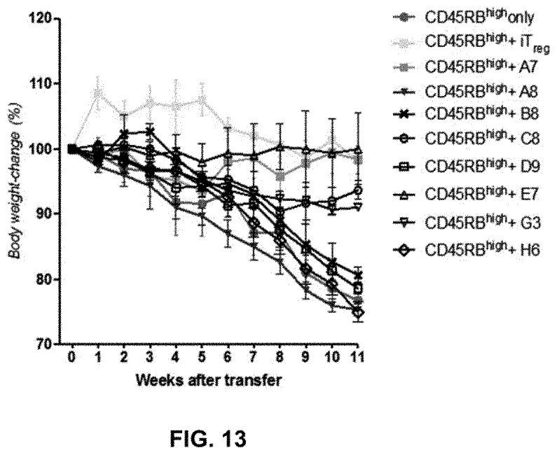

In order to identify therapeutic effects of the monoclonal antibodies (A7, C8, E7, and G3) according to the present invention produced in Production Examples 1 to 4 on an autoimmune disease, RAG-1^{-/-} mice were subjected to adoptive transfer with CD45RB (high) cells so that inflammatory bowel disease (IBD), which is an autoimmune disease, was induced. Then, the antibodies of Production Examples 1 to 4 were intraperitoneally injected in an amount of 200 μg/mouse, and then therapeutic effects thereof on the autoimmune disease were analyzed. The results are illustrated in FIG. 13.

As illustrated in FIG. 13, it was found that the Lrig-1 protein-specific monoclonal antibodies (A7, C8, E7, and G3) according to the present invention remarkably inhibit a body weight-decreasing effect in inflammatory bowel disease-induced mice.

From this, it can be seen that the Lrig-1 protein-specific monoclonal antibody according to the present invention are capable of effectively preventing, ameliorating, or treating immune-related diseases, such as autoimmune diseases, graft-versus-host diseases, organ transplant rejection, asthma, atopy, or acute or chronic inflammatory disease, which are induced by excessive activation and expression of various immune cells and inflammatory cells.

Although the present invention has been described in detail above, the scope of the present invention is not limited thereto. It will be obvious to those skilled in the art that various modifications and changes can be made without departing from the technical spirit of the present invention described in the claims.

### INDUSTRIAL APPLICABILITY

The present invention relates to a binding molecule capable of specifically binding to leucine-rich and immunoglobulin-like domains 1 (Lrig-1) protein, which is a protein present on the surface of regulatory T cells (Treg cells), and a use thereof, specifically, prevention or treatment of immune-related diseases, such as autoimmune diseases, graft-versus-host diseases, organ transplant rejection, asthma, atopy, or acute or chronic inflammatory diseases.

1-25. (canceled)

26. A binding molecule specific for leucine-rich and immunoglobulin like domains 1 (LRIG-1) protein, wherein the binding molecule is selected from the group consisting of:
(i) a binding molecule comprising a heavy chain variable region having the amino acid sequence as set forth in SEQ ID NO: 11, and a light chain variable region having the amino acid sequence as set forth in SEQ ID NO: 12;
(ii) a binding molecule comprising a heavy chain variable region having the amino acid sequence as set forth in SEQ ID NO: 19, and a light chain variable region having the amino acid sequence as set forth in SEQ ID NO: 20;
(iii) a binding molecule comprising a heavy chain variable region having the amino acid sequence as set forth in SEQ ID NO: 27, and a light chain variable region having the amino acid sequence as set forth in SEQ ID NO: 28; and
(iv) a binding molecule comprising a heavy chain variable region having the amino acid sequence as set forth in SEQ ID NO: 35, and a light chain variable region having the amino acid sequence as set forth in SEQ ID NO: 36.

27. The binding molecule of claim 26, wherein the LRIG-1 protein comprises the amino acid sequence as set forth in SEQ ID NO: 1 or 3.

28. The binding molecule of claim 27, wherein the LRIG-1 protein is encoded by a polynucleotide having the nucleotide sequence as set forth in SEQ ID NO: 2 or 4.

29. The binding molecule of claim 26, further comprising an Fc region or a constant region.

30. The binding molecule of claim 29, wherein the Fc region is an Fc region of an IgG1, IgG2, IgG3, or IgG4 antibody, or a hybrid Fc region.

31. The binding molecule of claim 26, further comprising a heavy chain constant region having the amino acid sequence as set forth in SEQ ID NOs: 37, 39, 41, 42, 43, 44, or 53.

32. The binding molecule of claim 26, further comprising a light chain constant region having the amino acid sequence as set forth in SEQ ID NO: 38 or 40.

33. The binding molecule of claim 26, further comprising a heavy chain constant region having the amino acid sequence as set forth in SEQ ID NO: 37, and a light chain constant region having the amino acid sequence as set forth in SEQ ID NO: 38.

34. The binding molecule of claim 26, further comprising a heavy chain constant region having the amino acid sequence as set forth in SEQ ID NO: 39, 41, 42, 43, or 53, and a light chain constant region having the amino acid sequence as set forth in SEQ ID NO: 40.

35. The binding molecule of claim 26, further comprising a heavy chain constant region having the amino acid sequence as set forth in SEQ ID NO: 44.

36. The binding molecule of claim 26, wherein the binding molecule is selected from the group consisting of:
(i) a binding molecule comprising a heavy chain having the amino acid sequence as set forth in SEQ ID NO: 45, and a light chain having the amino acid sequence as set forth in SEQ ID NO: 46;
(ii) a binding molecule comprising a heavy chain having the amino acid sequence as set forth in SEQ ID NO: 47, and a light chain having the amino acid sequence as set forth in SEQ ID NO: 48;
(iii) a binding molecule comprising a heavy chain having the amino acid sequence as set forth in SEQ ID NO: 49, and a light chain having the amino acid sequence as set forth in SEQ ID NO: 50; and
(iv) a binding molecule comprising a heavy chain having the amino acid sequence as set forth in SEQ ID NO: 51, and a light chain having the amino acid sequence as set forth in SEQ ID NO: 52.

37. The binding molecule of claim 26, wherein the binding molecule is an antibody or a fragment thereof.

38. The binding molecule of claim 37, wherein the antibody is a chimeric antibody, a humanized antibody, a bivalent, a bispecific molecule, a minibody, a domain antibody, a bispecific antibody, an antibody mimetic, a unibody, a diabody, a triabody, or a tetrabody, or a fragment thereof.

39. A pharmaceutical composition comprising the binding molecule of claim 26.

40. A method of preventing or treating an immune-related disease in a subject in need thereof, the method comprising administering a pharmaceutically effective amount of the binding molecule of claim 26 to the subject.

41. The method of claim 40, wherein the immune-related disease is an autoimmune disease, a graft versus host disease, an organ transplant rejection, asthma, atopy, or an acute or chronic inflammatory disease.

42. The method of claim 40, wherein the binding molecule is administered to the subject via oral, intravenous, intramuscular, intra-arterial, intramedullary, intradural, intracardiac, transdermal, subcutaneous, intraperitoneal, intranasal, intestinal, topical, sublingual, or rectal route.

43. A method of preventing or treating an immune-related disease in a subject in need thereof, the method comprising administering a pharmaceutically effective amount of the pharmaceutical composition of claim 39 to the subject.

44. The method of claim 43, wherein the immune-related disease is an autoimmune disease, a graft versus host disease, an organ transplant rejection, asthma, atopy, or an acute or chronic inflammatory disease.

45. The method of claim 43, wherein the pharmaceutical composition is administered to the subject via oral, intravenous, intramuscular, intra-arterial, intramedullary, intradural, intracardiac, transdermal, subcutaneous, intraperitoneal, intranasal, intestinal, topical, sublingual, or rectal route.
